(19) United States
(12) Patent Application Publication
(10) Pub. No.: US 2012/0244161 A1
(43) Pub. Date: Sep. 27, 2012

(54) DOSAGE REGIMEN FOR ADMINISTERING AN EpCAMxCD3 BISPECIFIC ANTIBODY

(76) Inventors: Gerhard Zugmeier, Munich (DE); Peter Kufer, Munich (DE); Dominik Ruttinger, Munich (DE); Sabine Kaubitzsch, Munich (DE)

(21) Appl. No.: 13/496,836

(22) PCT Filed: Sep. 20, 2010
(86) PCT No.: PCT/EP10/63795

§ 371 Date: Jun. 1, 2012

Related U.S. Application Data
(60) Provisional application No. 61/243,651, filed on Sep. 18, 2009.
(60) Provisional application No. 61/344,147, filed on Jun. 1, 2010.

(30) Foreign Application Priority Data
Sep. 18, 2009 (EP) 09170715.8
Jun. 1, 2010 (EP) 10164596.8

(51) Int. Cl. A61K 39/395; A61P 35/00

(52) U.S. Cl. 424/135.1; 424/136.1

(57) ABSTRACT

The present invention relates to a method (dosage regimen) for administering an EpCAMxCD3 bispecific antibody to a human patient, comprising (a) administering continually a first dose of said antibody for a first period of time; and consecutively (b) administering continually a second dose of said antibody for a second period of time, wherein said second dose exceeds said first dose. The methods of the invention (and likewise the dosage regimen of the invention) are also suitable for treating EpCAM positive epithelial cancer cells in a human patient, or for ameliorating and/or preventing a medical condition mediated by the continued administration of an EpCAMxCD3 bispecific antibody to a human patient. The present invention also relates to the use of an EpCAMxCD3 bispecific antibody for the preparation of a pharmaceutical composition to be used in a method as defined in any one of the preceding claims. A pharmaceutical package or kit comprising the first dose and the second dose as defined in the methods/dosage regimen of the present invention is disclosed as well.

The present invention relates to a method (dosage regimen) for administering an EpCAMxCD3 bispecific antibody to a human patient, comprising (a) administering continually a first dose of said antibody for a first period of time; and consecutively (b) administering continually a second dose of said antibody for a second period of time, wherein said second dose exceeds said first dose. The methods of the invention (and likewise the dosage regimen of the invention) are also suitable for treating EpCAM positive epithelial cancer cells in a human patient, or for ameliorating and/or preventing a medical condition mediated by the continued administration of an EpCAMxCD3 bispecific antibody to a human patient. The present invention also relates to the use of an EpCAMxCD3 bispecific antibody for the preparation of a pharmaceutical composition to be used in a method as defined in any one of the preceding claims. A pharmaceutical package or kit comprising the first dose and the second dose as defined in the methods/dosage regimen of the present invention is disclosed as well.

Antibody-based cancer therapies require a target antigen firmly bound to the surface of cancer cells in order to be active. By binding to the surface target, the antibody can deliver a deadly signal to the cancer cell. In an ideal treatment scenario, a target antigen is abundantly present and accessible on every cancer cell and is absent, shielded or much less abundant on normal cells. This situation provides the basis for a therapeutic window in which a defined amount of the antibody-based therapeutic effectively hits cancer cells but spares normal cells.

EpCAM is found on most human adenocarcinoma, including cancers of colorectal, breast, lung, gastric, bladder, prostate, ovarian, and pancreatic origin. For instance, in colorectal cancer, more than 98% of patients show an intense and frequent expression of EpCAM on cancer cells in the primary tumor (P. Went et al., Br. J. Cancer 94: 128 (2006)). EpCAM is not lost from cancer cells when they de-differentiate and progress to the metastatic stage. In some cancers, such as breast, ovarian and certain squamous cell carcinomas, EpCAM expression is either de novo or highly upregulated compared to normal epithelial tissues. When EpCAM expression is knocked down in cancer cells by anti-sense or siRNA, cells cease to proliferate, move and invasively grow in soft agar. Conversely, ectopic expression of EpCAM in quiescent cells confers these properties, and leads to their serum growth factor-independent growth (M. Münz et al., Oncogene 23: 5748 (2004)). EpCAM has now been added to the list of cancer stem cell markers (J. E. Visvader and G. J. Lindeman, Nat. Rev. Cancer 8: 755 (2008)). Cancer stem cells are thought to constantly repopulate tumors and to be responsible for chemoresistance and tumor relapse. EpCAM expression has been found on cancer stem cells derived from breast, colon, prostate, liver and pancreas tumors.

EpCAM is currently being targeted by several antibody-based therapeutic approaches, which are in different stages of clinical development. The following adverse events have been reported upon treatment of patients with these EpCAM antibodies.

### Catumaxomab:

Systemic cytokine release causing pyrexia, tachycardia
Decrease in lymphocytes
Increase in liver parameters with transaminases up to Grade 4 at high dose with low dexamethasone

### VB4-845

Mild fever, nausea, vomiting
Increase in transaminases

MT110 is a bispecific single chain antibody construct (BiTE) binding to epithelial cell adhesion molecule (EpCAM), expressed on most solid cancers of epithelial origin, and to CD3 on T cells. MT110 has shown high anti-tumor activity in various preclinical models including a human colorectal cancer (CRC) xenograft. Clinical proof of concept for BiTE antibodies has been demonstrated with blinatumomab (CD19xCD3 BiTE) in patients (pts) with B cell lymphoma (Bargou R et al. (2008) Science 321:9741). MT110 is presently under investigation in a dose-escalating phase 1 trial with (metastatic) gastrointestinal and lung cancer patients. In order to evaluate safety and tolerability of the anti-EpCAM×anti-CD3 bispecific single chain antibody, the compound has been administered by long-term continuous infusion. None of the patients developed fever, chills or other infusion reactions after start of the infusion. No substantial systemic cytokine levels could be found. However, a transient elevation of liver enzymes has been observed upon start of infusion of the EpCAMxCD3 bispecific single chain antibody.

Evidently, it is difficult to design an anti-EpCAM-antibody based therapy, which does not affect liver parameters like liver enzymes etc. of the treated patients.

Thus, the technical problem underlying the present invention was to provide methods to overcome the above problem.

The present invention addresses this need and thus provides embodiments concerning methods as well as dosage regimens for administering an EpCAMxCD3 bispecific antibody to a human patient.

These embodiments are characterized and described herein and reflected in the claims.

It must be noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

All publications and patents cited in this disclosure are incorporated by reference in their entirety. To the extent the material incorporated by reference contradicts or is inconsistent with this specification, the specification will supersede any such material. Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

In view of the adverse events, particularly the alarming increase in liver parameters such as ALT, AST, AP, etc. (the terms “AP. ALT and AST etc.” are explained herein elsewhere), observed with EpCAM specific antibodies, the finding that the EpCAMxCD3 bispecific single chain antibody is well tolerated by the patients once it is administered in accordance with the dosage regimen as provided herein, is definitely remarkable.

Specifically, the present inventors observed that the serum level of liver enzymes increases significantly to an extent which is undesired since it may be an additional burden for patients which are subject to a treatment with an EpCAM specific antibody such as an EpCAMxCD3 bispecific antibody. However, strikingly, the increase in transaminases did not occur on re-exposure to an EpCAMxCD3 bispecific antibody, provided that the antibody was administered in accordance with the methods/dosage regimen as disclosed herein. In sum, the present inventors found that “adapting” a patient to an EpCAMxCD3 bispecific antibody prior to the therapy with an EpCAMxCD3 bispecific antibody is beneficial for avoiding undesired adverse effect (particularly the unwanted increase in liver parameters).

Accordingly, the present invention relates in a first aspect to a method (dosage regimen) for administering an EpCAMxCD3 bispecific antibody to a human patient, comprising:
(a) administering continually a first dose of said antibody for a first period of time; and consecutively
(b) administering continually a second dose of said antibody for a second period of time;
   wherein said second dose exceeds said first dose.

It is preferred that in the context of the present invention the human patient comprises or is assumed to comprise EpCAM positive epithelial cancer cells.

It will be understood that in the context of the present invention, the term “method” includes a “dosage regimen” to be used in a method of the present invention.

In the context of the present invention “administration of an EpCAMxCD3 bispecific antibody” or “administering an EpCAMxCD3 bispecific antibody” or any other grammatical form thereof means that the EpCAMxCD3 antibody is in the form of a pharmaceutical composition, optionally comprising a pharmaceutically acceptable carrier. Accordingly, it is to be understood that a pharmaceutical composition comprising an EpCAMxCD3 bispecific antibody is administered to a human patient.

In the context of the present invention the term “patient” means a subject or individual in need of a treatment of EpCAM positive epithelial cancer cells. The patient is a mammal, preferably a human.

The term “administering” in all of its grammatical forms means administration of an EpCAMxCD3 bispecific antibody (in the form of a pharmaceutical composition) either as the sole therapeutic agent or in combination with another therapeutic agent.

It is thus envisaged that the pharmaceutical composition of the present invention are also employed in co-therapy approaches, i.e. in co-administration with other medicaments or drugs, for example, other medicaments for treating EpCAM positive epithelial cancer cells in a human patient and/or with glucocorticoids and/or any other therapeutic agent which might be beneficial in the context of the methods of the present invention.

The administration of a pharmaceutical composition referred to herein is preferably an intravenous administration. It follows that in the methods of the present invention the route of administration in step (a) and/or the route of administration in step (b) is intravenous.

The administration of an EpCAMxCD3 bispecific antibody (for example in the form of a pharmaceutical composition) is continually or as also used herein continuously. A continual administration refers to an administration which is essentially without interruption. “Essentially without interruption” includes a continual administration usually without an uninterrupted flow or spatial extension.

In a preferred embodiment of the present invention the second dose is therapeutically active. Preferably, an active dose effects activation of CD8+-T cells. Activated CD8+-T cells are preferably characterized by a CD25 and/or CD69 phenotype. The term “activated CD8+-T cells” and “CD25 and/or CD69 phenotype” are described herein elsewhere.

By “therapeutically effective amount” or “therapeutic active” is meant a dose of an EpCAMxCD3 bispecific antibody that produces the therapeutic effects for which it is administered.

The exact dose will depend on the purpose of the treatment, and will be ascertainable by one skilled in the art using known techniques. As is known in the art and described above, adjustments for age, body weight, general health, sex, diet, drug interaction and the severity of the condition may be necessary, and will be ascertainable with routine experimentation by those skilled in the art. “Therapeutically active” includes in the context of the present invention at least the above mentioned activation of CD8+-T cells, which is a prerequisite for a therapeutic approach. The therapeutic effect of the respective methods or method steps of the present invention is additionally detectable by all established methods and approaches which will indicate a therapeutic effect. It is, for example, envisaged that the therapeutic effect is detected by way of surgical resection or biopsy of an affected tissue/organ which is subsequently analyzed by way of immunohistochemical (IHC) or comparable immunological techniques. Alternatively it is also envisaged that the tumor markers in the serum of the patient (if present) are detected in order to diagnose whether the therapeutic approach is already effective or not. Additionally or alternatively it is also possible to evaluate the general appearance of the respective patient (fitness, well-being, decrease of tumor-mediated ailment etc.) which will also aid the skilled practitioner to evaluate whether a therapeutic effect is already there. The skilled person is aware of numerous other ways which will enable him or her to observe a therapeutic effect of the compounds of the present invention.

In a further aspect, the present invention relates to a method for treating EpCAM positive epithelial cancer cells in a human patient, said method comprising:
(a) administering continually a first dose of an EpCAMxCD3 bispecific antibody for a first period of time; and consecutively
(b) administering continually a second dose of said antibody for a second period of time;
   wherein said second dose exceeds said first dose.

In a still further aspect, the present invention relates to a method for ameliorating and/or preventing a medical condition, preferably an adverse effect, mediated by the continued (therapeutic) administration of an EpCAMxCD3 bispecific antibody to a human patient, said method comprising:
(a) administering continually a first dose of said antibody for a first period of time, and consecutively
(b) administering continually a second dose of said antibody for a second period of time;
   wherein said second dose exceeds said first dose. It is preferred that said human patient comprises or is assumed to comprise EpCAM positive epithelial cancer cells.

In a preferred embodiment of the method for ameliorating or preventing a medical condition, preferably an adverse effect, mediated by the administration of an EpCAMxCD3 bispecific antibody to a human patient, said medical condition is characterized by an increase of the serum level of at least one liver enzyme.

The increase of the serum level of said at least one liver enzyme is at its maximum up to grade 4 in accordance with the Common Terminology Criteria for Adverse Events v3.0 (CTCAE) which is further described herein below. Accordingly, the increase of the serum level of said at least one liver enzyme may also be up to grade 1, 2 or 3, while grade 4 is the maximum.

The increase is preferably a transient one. “Transient” when used in the context of an increase of the serum level of said at least one liver enzyme means that the increase is not permanent, but disappears after treatment stop or during continued further infusion. It is also envisaged that the transient increase in the serum level of liver enzymes is not necessarily accompanied by pathological findings in imaging, substantial tissue damage or impaired synthesis parameters of the liver. This is exemplarily shown in FIG. 7 which depicts the liver parenchyma of a patient who was treated with MT110, i.e. an antibody of the invention. As can be seen, the liver parenchyma shows no signs of substantial cellular damage (HE staining) at peak of liver transaminases.

Generally spoken, the activity of a liver enzyme is commonly used as a “window” to the liver, since it provides guidance about the condition/state of the liver. For example, if the liver is damaged by, for example, alcohol or other medicaments, or has an abnormal function for any other reason, liver enzymes leak into the blood where they are normally not present.

Accordingly, the serum level of a liver enzyme can be measured by way of the activity of the liver enzyme. An activity of a liver enzyme that is above (i.e. increased or elevated) a commonly accepted reference value is usually indicative of a potential abnormal function and/or damage of the liver.

An activity of a liver enzyme is thus measurable by liver function tests (LFTs or LFs), i.e., clinical biochemistry laboratory blood assays designed to give information about the state of a patient's liver. For liver enzymes, reference values (normal values) are known and commonly accepted. A reference value is a set of values used by a health professional to interpret a set of medical test results. The reference value is usually defined as the set of values 95 percent of the normal population falls within, or two standard deviations from the mean. It is determined by collecting data from vast numbers of laboratory tests.

In case of liver enzymes the reference value is given as international units (IU). International units are based on measured biological activity or effect.

An increase (preferably transient) of the serum level of a liver enzyme is measured in multiples of the upper limit of normal (ULN). In accordance with the NCI Common Terminology Criteria for Adverse Events v3.0 (CTCAE) (Publish Date: Dec. 12, 2003) the multiples of the ULN are categorized in grades. A Grade refers to the severity of the adverse effects. The CTCAE v3.0 displays grades 1 through 5 with unique clinical descriptions of severity for each adverse effects:

Grade 1: mild adverse effects
Grade 2: Moderate adverse effects
Grade 3: Severe adverse effects
Grade 4: Life-threatening or disabling adverse effects.
Grade 5: Death of the patient

Liver transaminases such as aspartate transaminase (AST) and alanine transaminase (ALT) provide for the state of cellular integrity of the liver, since in case of a liver damage or malfunction these enzymes leak from damaged or malfunctioning liver cells into the blood.

Accordingly, in the context of the methods of the present invention. AST and/or ALT are the preferred liver enzymes (liver markers). In some preferred embodiments said at least one liver enzyme comprises AST and/or ALT and optionally also GGT and/or AP.

Aspartate transaminase (AST) also called Serum Glutamic Oxaloacetic Transaminase (SGOT) or aspartate aminotransferase (ASAT) is similar to Alanine transaminase (ALT) in that it is another enzyme associated with liver parenchymal cells. It is raised in acute liver damage, but is also present in red blood cells, and cardiac and skeletal muscle and is therefore not specific to the liver. The ratio of AST to ALT is sometimes useful in differentiating between causes of liver damage. Elevated AST levels are not specific for liver damage. A usual reference value for AST is 10 to 50 IU/I.

In accordance with the Common Terminology Criteria for Adverse Events v3.0 the grading for AST is as follows:

| Grade 1 | Grade 2 | Grade 3 | Grade 4 | Grade 5 |
|---|---|---|---|---|
| >ULN-2.5 × | >2.5-5.0 × | 5.0-20.0 × | >20.0 × | — |
| ULN | ULN | ULN | ULN | |

Alanine transaminase (ALT), also called Serum Glutamic Pyruvate Transaminase (SGPT) or Alanine aminotransferase (ALAT) is an enzyme present in hepatocytes (liver cells). When a cell is damaged, it leaks this enzyme into the blood, where it is measured. ALT rises dramatically in acute liver damage, such as viral hepatitis or paracetamol (acetaminophen) overdose. A usual reference value for ALT is 5 to 50 IU/I.

In accordance with the Common Terminology Criteria for Adverse Events v3.0 the grading for ALT is as follows:

| Grade 1 | Grade 2 | Grade 3 | Grade 4 | Grade 5 |
|---|---|---|---|---|
| >ULN-2.5 × | >2.5-5.0 × | >5.0-20.0 × | >20.0 × | — |
| ULN | ULN | ULN | ULN | |

Other liver enzymes such as gamma-glutamyl transferase (GGT) or alkaline phosphatase (AP) provide for conditions linked to the biliary tract.

Accordingly, it is also envisaged that as a further liver enzyme alkaline phosphatase (AP or ALP) the increase or decrease, respectively, could be measured in the context of the present invention. AP is an enzyme in the cells lining the biliary ducts of the liver. AP levels in plasma will rise with large bile duct obstruction, intrahepatic cholestasis or infiltrative diseases of the liver. AP is also present in bone and placental tissue, so it is higher in growing children (as their bones are being remodelled) and elderly patients with Paget's disease. A usual reference value for AP is 30 to 120 IU/I.

In accordance with the Common Terminology Criteria for Adverse Events v3.0 the grading for AP is as follows:

| Grade 1 | Grade 2 | Grade 3 | Grade 4 | Grade 5 |
|---|---|---|---|---|
| >ULN-2.5 × | >2.5-5.0 × | >5.0-20.0 × | >20.0 × | — |
| ULN | ULN | ULN | ULN | |

Another liver enzyme the increase or decrease, respectively, could be measured in the context of the present invention is gamma glutamyl transpeptidase (GGT), It is known to be elevated with even minor, sub-clinical levels of liver dysfunction, It can also be helpful in identifying the cause of an isolated elevation in ALP, A usual reference value for GGT is 0 to 51 IU/I.

In accordance with the Common Terminology Criteria for Adverse Events v3.0 the grading for GGT is as follows:

| Grade 1 | Grade 2 | Grade 3 | Grade 4 | Grade 5 |
|---|---|---|---|---|
| >ULN-2.5 × | >2.5-5.0 × | >5.0-20.0 × | >20.0 × | — |
| ULN | ULN | ULN | ULN | |

In one aspect of the methods of the present invention said second period of time exceeds said first period of time. The term “exceeds” means that the second period of time is longer than the first period of time, preferably at least one day longer. In another aspect of the methods/dosage regimen of the present invention, said first period of time exceeds or equates said second period of time.

It is also envisaged that the first period of time and the second period of time are interrupted by a third period of time (i.e. a break between the first period of time and the second period of time). Said third period of time is preferably as short as possible (as the EpCAM-positive epithelial cancer might grow in the meantime) but may last for one or more days or even one or two or even more weeks depending on the circumstances. The aim of this interruption has to be seen as a recreational phase allowing the patient to recover from the increase of the serum level of said at least one liver enzyme, provided that this is necessary. In a preferred embodiment said third period of time (which is between the first and the second period of time) is two weeks or less, more preferably it is one week or less.

In another aspect of the present invention, it is envisaged that said first period of time is at least 1, 2, 3, 4, 5, 6, 7 days long, whereby even longer periods of time of for example 8, 9, 10, 11, 12, 13 or 14 days are not excluded. “Longer” is thereby not limited to a (one) complete day as the lowest time unit, i.e. ½ days, or fully hours are also conceivable. It is however preferred that the smallest time unit is one full day. In view of the results shown in the appended examples, it turns out that the first period of time is ideally between 7 and 9 days (7, 8, 9d), 7 days being preferred. Preferably said EpCAMxCD3 bispecific antibody is administered in that first period of time such that the serum level of at least one liver enzyme is increased to grade 4 or less (preferably to grade 3) and subsequently decreased to grade 2. This increase and decrease of a liver enzyme has been explained herein elsewhere.

As used herein, a time interval which is defined as “X to Y” equates with a time interval which is defined as “between X and Y”. Both time intervals specifically include the upper limit and also the lower limit. This means that for example a time interval “1 to 4 days” or between “1 to 4 days” includes a period of time of one, two, three and/or four days.

As mentioned herein, the present inventors observed that “adapting” a human patient to the treatment with an EpCAMxCD3 bispecific antibody during a first period of time allows the treatment of the human patient with an increased second dose of the antibody for a second period of time, whereby adverse effects (increase of the serum level of at least one liver enzyme) can be better controlled, i.e., kept within an acceptable grade in accordance with the CTCAE.

However, for achieving this improvement it is required to “adapt” the human patient to the EpCAMxCD3 bispecific antibody by continually administering a first dose of the antibody for a first period of time (wherein said first dose is lower than the consecutive (second) dose).

That first period of time, in which a first dose of an EpCAMxCD3 bispecific antibody is continually administered to a human patient, is preferably characterized by an increase of the serum level of at least one liver enzyme up to grade 3 or 4 (thus including an increase of the serum level of at least one liver enzyme to grade 1 or 2). The increase is seen in relation to the serum level of said at least one liver enzyme at the start of the first period.

The “adaptation” phase (which includes the first period of time in which a first dose of an EpCAMxCD3 bispecific antibody is continually administered to a human patient) preferably persists until the increased serum level of said at least one liver enzyme is decreased to preferably grade 2 or even grade 1, Said adaptation phase may also include the above mentioned third period of time (provided that a third period of time is employed) which third period of time represents a recreational break allowing the patient to recover from the increase of the serum level of at least one liver enzyme (if necessary).

In sum, the first period of time in which a first dose of an EpCAMxCD3 bispecific antibody is continually administered to a human patient is characterized by an increase of the serum level of at least one liver enzyme and by a decrease of the serum level of at least one liver enzyme as described before. Said decrease may also take place during the third period of time, provided that a third period of time is employed. This increase and decrease thus characterizes the adaptation phase of a human patient to whom an EpCAMxCD3 bispecific antibody is to be administered, thereby allowing the continual administration of a second dose of an EpCAMxCD3 bispecific antibody for a second period of time without having an excess increase of the serum level of at least one liver enzyme.

Accordingly, the first period of time (or first and third period of time) is dependent on the time required for the increase and decrease and may thus vary from patient to patient. However, the skilled practitioner by applying the teaching of the present invention is readily in a position to determine the increase of the serum level of at least one liver enzyme by determining the serum level and comparing it to the serum level at the start of the “adaptation phase”, i.e., at the start of the first period of time (and third period of time, if present) in which a first dose of an EpCAMxCD3 bispecific antibody is continually administered to a human patient.

Likewise, the skilled practitioner is readily in a position to determine the decrease of the serum level of at least one liver enzyme by determining the serum level after it has reached a maximum grade of 4 and evaluating whether it is then decreased.

In a particular preferred embodiment of the methods of the present invention the first period of time in which an EpCAMxCD3 bispecific antibody is continually administered to a human patient is equal to or less than 4 days (for example 3 days), provided that the serum level of said at least one liver enzyme does not increase during said period of time above grade 2.

Likewise the duration of the first period of time, the duration of the second period of time and the duration of the third period of time may be variable in view of, for example, the age, sex, body weight, etc. of the human patient.

The second period of time preferably persists until the CD8+-T-cells of said patient are being activated.

Activation of CD8+-T cells can be determined by means and methods known in the art, such as FACS-analysis by applying antibodies for CD8, thereby sorting for CD8+-T cells and/or by applying antibodies specific for cell surface markers which are indicative for the activation of CD8+-T cells.

Accordingly. CD8+-T cell activation is preferably characterized by an increase of a CD25 and/or CD69-positive phenotype of at least 20%, 30%, 40%, 50% or more of said CD8+-T-cells. The total number of CD8+-T cells can be determined by means and methods known in the art in, for example, by FACS-analysis of a sample of peripheral blood of said patient. Said increase during the second period is to be seen in relation to the CD25 and/or CD69-positive phenotype of CD8+-T cells of the patient prior to the first period or prior to the second period. Accordingly, it is envisaged that the phenotype of CD8+-T cells of the patient is determined prior to the first period or prior to the second period in order to have available a reference value.

In another embodiment of the methods of the present invention, said second period of time is at least 2 weeks, i.e. 2, 3, 4, 5, 6, 7, 8 or even more weeks, 3 weeks being preferred. It will be understood that the term “one week” means seven full days.

In a preferred embodiment, said second period of time is at least 19 days, i.e. 19, 20, 21, 22, 23, 24, 25, 26, 27, 28, 29, 30, or even more days. It is preferred that said second period of time persists up to the detection of a therapeutic effect. “Therapeutic effect” includes in the context of the present invention at least the above mentioned activation of CD8+-T cells, which is a prerequisite for any therapeutic effect.

In a more preferred embodiment of the methods/dosage regimens of the present invention, said first period of time is between 1 and 10 days, and that second period of time is at least 19 days.

In an even more preferred embodiment, said first period of time is 7 to 9 days and that second period of time is 19 to 21 days. Particularly preferred is a first period of time of 7 days and a second period of time of 21 days. In another particularly preferred embodiment, said first period of time is one week, said second period of time is at least three weeks and that third period of time is one or two weeks.

In some embodiments, said first dose is not therapeutically active. “Not therapeutically active” means in this context that the above described CD8+-T cell activation is preferably characterized by an increase of a CD25 and/or CD69-positive phenotype of CD8+-T cell of 20%, or below. The total number of CD8+-T cells can be determined by means and methods known in the art in, for example, by FACS-analysis of a sample of peripheral blood of said patient. Said increase is to be seen in relation to the CD25 and/or CD69-positive phenotype of CD8+-T cells of the patient prior to the first period. Accordingly, it is envisaged that the phenotype of CD8+-T cells of the patient is determined prior to the first period in order to have available a reference value.

It is envisaged that second dose is therapeutically active. Therapeutic activity of said second dose is characterized by activated CD8+-T-cells as described herein elsewhere. Said activation is characterized by a CD25 and/or CD69-positive phenotype of more than 20% of said CD8+-T-cells (in relation to the CD25 and/or CD69-positive phenotype prior to the second period).

In another embodiment of the methods of the present invention, said first dose is such that the serum level of at least one liver enzyme increases to a serum level of grade 3 or 4 and decreases again to a serum level of grade 2 within the first period of time. The terms “liver enzyme”, “grade” etc. are described elsewhere herein.

In a further aspect of the methods/dosage regimens of the present invention, said first dose is 1 to 6 μg/d, i.e. 1, 2, 3, 4, 5, or 6 μg/d (1 to 3 μg/d, i.e. 1, 2, or 3 μg/d being preferred). “d” denotes one day. A dose of for example, 1 μg/d means that 1 μg of the EpCAMxCD3 bispecific antibody is administered evenly or continuously across one day. “Continuously across one day” refers to an infusion which is allowed to proceed permanently without interruption. It is also envisaged that the first dose is increased over time during said first period of time (i.e. the first period of time is split up into several steps which are characterized by an increase of the dose), wherein said increase ends up at a dose which as such is below said second dose. Said stepwise increase within the first period of time might occur from day to day or from week to week (see for example FIG. 8). It is thus envisaged that the first period of time starts with a “dose escalation” treatment/dosage regimen which is characterized by a stepwise adaptation of the patient to the treatment (for example one week 3 μg/day followed by one week 6 or 12 μg/day).

In a further aspect of the methods/dosage regimen of the present invention, said second dose is 10 to 120 μg/d, i.e. 10, 20, 30, 40, 50, 60, 70, 80, 90, 100, 110, or 120 μg/d, or even more.

The term “μg” includes “μg of the EpCAMxCD3 bispecific antibody preparation”. It is preferred that not more than 10% of said EpCAMxCD3 bispecific antibody preparation is incorrectly folded. It follows that in a preferred embodiment, 90%, 91%, 92%, 93%, 94%, 95%, 96%, 97%, 98%, 99% or even 100% of the EpCAMxCD3 bispecific antibody is correctly folded. It is also conceivable that the antibody preparation may optionally comprise further ingredients, for example a lyoprotectant, a surfactant, a filler, a binder, and/or bulking agent etc. The amount of such further ingredients is, preferably, not included in the term “μg” as used in the context of the “dose” and or methods of the present invention.

In a preferred embodiment, said first dose is 1 to 3 μg/d (i.e. 1, 2, or 3) and that second dose is 20 to 90 μg/d (i.e. 20, 30, 40, 50, 60, 70, 80, or 90).

It must be understood that the ranges given herein are illustrated by increments of ten. These ranges, however, also encompass smaller increments, for example those exemplified by increments of one (10 to 30 includes for example 10, 11, 12, 13, 13 etc. up to 30), or still smaller increments, for example values after the decimal point.

In a preferred embodiment, said dosage regimen is as depicted in FIG. 8, Further details to these dosage regimens are depicted in Example 4 or 5 which comprises embodiments of the present invention.

As noted herein above, the present invention methods of treatment/dosage regimen which employ EpCAMxCD3 bispecific antibodies, comprising a first binding domain capable of binding to an epitope of human CD3 epsilon chain and a second binding domain capable of binding to human EpCAM. Examples for bispecific molecules according to the methods of the invention are described in great detail in WO2005/040220 (PCT/EP2004/011646), which is incorporated herein by reference in its entirety. All the specific EpCAMxCD3 bispecific antibodies disclosed therein, including their variants, fragments, equivalents etc. are particularly preferred EpCAMxCD3 bispecific antibodies of the present invention.

As used herein, an “EpCAMxCD3 bispecific single chain antibodies” denotes a single polypeptide chain comprising two binding domains. Such single chain antibodies are preferred in the context of the methods/dosage regimen of the present invention. Each binding domain comprises at least one variable region from an antibody heavy chain (“VH or H region”), wherein the VH region of the first binding domain specifically binds to the CD3 epsilon molecule, and the VH region of the second binding domain specifically binds to EpCAM. The two binding domains are optionally linked to one another by a short polypeptide spacer. A non-limiting example for a polypeptide spacer is Gly-Gly-Gly-Gly-Ser (G-G-G-G-S) and repeats thereof. Each binding domain may additionally comprise one variable region from an antibody light chain (“VL or L region”), the VH region and VL region within each of the first and second binding domains being linked to one another via a polypeptide linker, for example of the type disclosed and claimed in EP 623679 B1, but in any case long enough to allow the VH region and VL region of the first binding domain and the VH region and VL region of the second binding domain to pair with one another such that, together, they are able to specifically bind to the respective first and second binding domains. Such EpCAMxCD3 bispecific single chain antibodies are described in great detail in WO2005/040220 (PCT/EP2004/011646), which is incorporated herein by reference in its entirety.

The term “binding domain” characterizes in connection with the present invention a domain of a polypeptide which specifically binds to/interacts with a given target structure/antigen/epitope. Thus, the binding domain is an “antigen-interaction-site”. The term “antigen-interaction-site” defines, in accordance with the present invention, a motif of a polypeptide, which is able to specifically interact with a specific antigen or a specific group of antigens, e.g. the identical antigen in different species. Said binding/interaction is also understood to define a “specific recognition”. The term “specifically recognizing” means in accordance with this invention that the antibody molecule is capable of specifically interacting with and/or binding to at least two, preferably at least three, more preferably at least four amino acids of an antigen, e.g. the human CD3 antigen as defined herein. Such binding may be exemplified by the specificity of a “lock-and-key-principle”. Thus, specific motifs in the amino acid sequence of the binding domain and the antigen bind to each other as a result of their primary, secondary or tertiary structure as well as the result of secondary modifications of said structure. The specific interaction of the antigen-interaction-site with its specific antigen may result as well in a simple binding of said site to the antigen. Moreover, the specific interaction of the binding domain/antigen-interaction-site with its specific antigen may alternatively result in the initiation of a signal, e.g. due to the induction of a change of the conformation of the antigen, an oligomerization of the antigen, etc. A preferred example of a binding domain in line with the present invention is an antibody. The binding domain may be a monoclonal or polyclonal antibody or derived from a monoclonal or polyclonal antibody.

The term “antibody” comprises derivatives or functional fragments thereof which still retain the binding specificity. Techniques for the production of antibodies are well known in the art and described, e.g. in Harlow and Lane “Antibodies, A Laboratory Manual”, Cold Spring Harbor Laboratory Press, 1988 and Harlow and Lane “Using Antibodies: A Laboratory Manual” Cold Spring Harbor Laboratory Press, 1999. The term “antibody” also comprises immunoglobulins (Ig's) of different classes (i.e. IgA, IgG, IgM, IgD and IgE) and subclasses (such as IgG1, IgG2 etc.).

The definition of the term “antibody” also includes embodiments such as chimeric, single chain and humanized antibodies, as well as antibody fragments, like, inter alia. Fab fragments. Antibody fragments or derivatives further comprise F(ab′)2, Fv, scFv fragments or single domain antibodies, single variable domain antibodies or immunoglobulin single variable domain comprising merely one variable domain, which might be VH or VL, that specifically bind to an antigen or epitope independently of other V regions or domains; see, for example. Harlow and Lane (1988) and (1999), loc. cit. Such immunoglobulin single variable domain encompasses not only an isolated antibody single variable domain polypeptide, but also larger polypeptides that comprise one or more monomers of an antibody single variable domain polypeptide sequence.

As used herein. CD3 epsilon denotes a molecule expressed as part of the T cell receptor and has the meaning as typically ascribed to it in the prior art. In human, it encompasses in individual or independently combined form all known CD3 subunits, for example CD3 epsilon, CD3 delta, CD3 gamma, CD3 zeta, CD3 alpha and CD3 beta. The human CD3 epsilon is indicated in GenBank Accession No. NM_{—}000733.

The human EpCAM is indicated in GenBank Accession No. NM_{—}002354.

In a preferred embodiment of the methods/dosage regimen of the present invention, the bispecific single chain antibody construct is an EpCAM VL-EpCAM VH-CD3 VH-CD3 VL bispecific single chain antibody construct.

In a more preferred embodiment of the methods/dosage regimen of the present invention, the bispecific single chain antibody construct comprises CDR H1-3 as disclosed in SEQ ID NO. 88 (CDRH1), SEQ ID NO 92 (CDRH2), and SEQ ID NO 96 (CDRH3) disclosed in WO2005/040220 (PCT/EP2004/011646), and CDR L1-3 as disclosed in SEQ ID NO. 100 (CDRL1), SEQ ID NO 102 (CDRL2), and SEQ ID NO 104 (CDRL3) as disclosed in WO2005/040220 (PCT/EP2004/011646), i.e. the CDRs which characterize MT110.

Said SEQ IDs are also depicted in the Table below:

| | |
|---|---|
| SEQ ID | <400> 88 |
| NO. 1 | Gly Tyr Thr Phe Thr Arg Tyr Thr Met His |
| (CDRH1) | 1 5 10 |
| SEQ ID | <400> 92 |
| NO 2 | Tyr Ile Asn Pro Ser Arg Gly Tyr Thr Asn Tyr Ala Asp Ser Val Lys Gly |
| (CDRH2) | 1 5 10 15 |
| SEQ ID | <400> 96 |
| NO 3 | Tyr Tyr Asp Asp His Tyr Cys Leu Asp Tyr |
| (CDRH3) | 1 5 10 |
| SEQ ID | <400> 100 |
| NO. 4 | Arg Ala Ser Gln Ser Val Ser Tyr Met Asn |
| (CDRL1) | 1 5 10 |
| SEQ ID | <400> 102 |
| NO 5 | Asp Thr Ser Lys Val Ala Ser |
| (CDRL2) | 1 5 |
| SEQ ID | <400> 104 |
| NO 6 | Gln Gln Trp Ser Ser Asn Pro Leu Thr |
| (CDRL3) | 1 5 |

In an even more preferred embodiment of the method of the invention, the EpCAMxCD3 bispecific single chain antibody construct comprises (or consists of) an amino acid sequence as set forth in SEQ ID NO. 63 as disclosed in WO2005/040220 (PCT/EP2004/011646) and depicted below, or an amino acid sequence at least 90%, preferably 95% identical to said SEQ ID NO. 63 (which is also disclosed herein below). The EpCAMxCD3 bispecific single chain antibody construct which is characterized by that sequence is MT110.

Amino acid sequence of MT110 (SEQ ID NO: 7)

```
<223> 5-10(VL-VH)xanti-CD3(VH(5)-VL(2))
<400> 63
Met Gly Trp Ser Cys Ile Ile Leu Phe Leu Val Ala Thr Ala Thr Gly
1 5 10 15
Val His Ser Glu Leu Val Met Thr Gln Ser Pro Ser Ser Leu Thr Val
                        20 25 30
Thr Ala Gly Glu Lys Val Thr Met Ser Cys Lys Ser Ser Gln Ser Leu
                35 40 45
Leu Asn Ser Gly Asn Gln Lys Asn Tyr Leu Thr Trp Tyr Gln Gln Lys
        50 55 60
Pro Gly Gln Pro Pro Lys Leu Leu Ile Tyr Trp Ala Ser Thr Arg Glu
65 70 75 80
Ser Gly Val Pro Asp Arg Phe Thr Gly Ser Gly Ser Gly Thr Asp Phe
                                85 90 95
Thr Leu Thr Ile Ser Ser Val Gln Ala Glu Asp Leu Ala Val Tyr Tyr
                        100 105 110
Cys Gln Asn Asp Tyr Ser Tyr Pro Leu Thr Phe Gly Ala Gly Thr Lys
                115 120 125
Leu Glu Ile Lys Gly Gly Gly Gly Ser Gly Gly Gly Gly Ser Gly Gly
        130 135 140
Gly Gly Ser Glu Val Gln Leu Leu Glu Gln Ser Gly Ala Glu Leu Val
145 150 155 160
Arg Pro Gly Thr Ser Val Lys Ile Ser Cys Lys Ala Ser Gly Tyr Ala
                                165 170 175
Phe Thr Asn Tyr Trp Leu Gly Trp Val Lys Gln Arg Pro Gly His Gly
                        180 185 190
Leu Glu Trp Ile Gly Asp Ile Phe Pro Gly Ser Gly Asn Ile His Tyr
                195 200 205
Asn Glu Lys Phe Lys Gly Lys Ala Thr Leu Thr Ala Asp Lys Ser Ser
        210 215 220
Ser Thr Ala Tyr Met Gln Leu Ser Ser Leu Thr Phe Glu Asp Ser Ala
225 230 235 240
Val Tyr Phe Cys Ala Arg Leu Arg Asn Trp Asp Glu Pro Met Asp Tyr
                                245 250 255
Trp Gly Gln Gly Thr Thr Val Thr Val Ser Ser Gly Gly Gly Gly Ser
                        260 265 270
Asp Val Gln Leu Val Gln Ser Gly Ala Glu Val Lys Lys Pro Gly Ala
                275 280 285
Ser Val Lys Val Ser Cys Lys Ala Ser Gly Tyr Thr Phe Thr Arg Tyr
        290 295 300
Thr Met His Trp Val Arg Gln Ala Pro Gly Gln Gly Leu Glu Trp Ile
305 310 315 320
Gly Tyr Ile Asn Pro Ser Arg Gly Tyr Thr Asn Tyr Ala Asp Ser Val
                                325 330 335
Lys Gly Arg Phe Thr Ile Thr Thr Asp Lys Ser Thr Ser Thr Ala Tyr
                        340 345 350
Met Glu Leu Ser Ser Leu Arg Ser Glu Asp Thr Ala Thr Tyr Tyr Cys
                355 360 365
Ala Arg Tyr Tyr Asp Asp His Tyr Cys Leu Asp Tyr Trp Gly Gln Gly
        370 375 380
Thr Thr Val Thr Val Ser Ser Gly Glu Gly Thr Ser Thr Gly Ser Gly
385 390 395 400
Gly Ser Gly Gly Ser Gly Gly Ala Asp Asp Ile Val Leu Thr Gln Ser
                                405 410 415
Pro Ala Thr Leu Ser Leu Ser Pro Gly Glu Arg Ala Thr Leu Ser Cys
                        420 425 430
Arg Ala Ser Gln Ser Val Ser Tyr Met Asn Trp Tyr Gln Gln Lys Pro
                435 440 445
Gly Lys Ala Pro Lys Arg Trp Ile Tyr Asp Thr Ser Lys Val Ala Ser
        450 455 460
Gly Val Pro Ala Arg Phe Ser Gly Ser Gly Ser Gly Thr Asp Tyr Ser
465 470 475 480
Leu Thr Ile Asn Ser Leu Glu Ala Glu Asp Ala Ala Thr Tyr Tyr Cys
                                485 490 495
Gln Gln Trp Ser Ser Asn Pro Leu Thr Phe Gly Gly Gly Thr Lys Val
                        500 505 510
Glu Ile Lys
                515
```

Thus, in a most preferred embodiment of the methods/dosage regimen of the present invention said EpCAMxCD3 bispecific single chain antibody is MT110 as characterized by the above indicated amino acid sequence.

The invention describes an EpCAMxCD3 bispecific single chain antibody molecule comprising an amino acid sequence as depicted in SEQ ID NO. 7 above (or in WO2005/040220 (PCT/EP2004/011646), as well as an amino acid sequence at least 90% or preferably 95% identical, most preferred at least 96, 97, 98, or 99% identical to said amino acid sequence. It is to be understood that the sequence identity is determined over the entire amino acid sequence. For sequence alignments, for example, the programs Gap or BestFit can be used (Needleman and Wunsch J. Mol. Biol. 48 (1970), 443-453; Smith and Waterman, Adv. Appl. Math 2 (1981), 482-489), which is contained in the GCG software package (Genetics Computer Group, 575 Science Drive, Madison, Wis., USA 53711 (1991). It is a routine method for those skilled in the art to determine and identify an amino acid sequence having e.g. 90%, 95%, 96%, 97%, 98% or 99% sequence identity to the amino acid sequences of the EpCAMxCD3 bispecific single chain antibody described herein (preferably MT110). For example, according to Crick's Wobble hypothesis, the 5′ base on the anti-codon is not as spatially confined as the other two bases, and could thus have non-standard base pairing. Put in other words: the third position in a codon triplet may vary so that two triplets which differ in this third position may encode the same amino acid residue. Said hypothesis is well known to the person skilled in the art (Crick, J Mol Biol 19 (1966): 548-55). It is furthermore a routine procedure for those skilled in the art to determine cytotoxic activity of such an amino acid sequence having e.g. 90%, 95%, 96%, 97%, 98% or 99% sequence identity to the nucleotide or amino acid sequences of the EpCAMxCD3 bispecific single chain antibody described herein. Cytotoxic activity of the EpCAMxCD3 bispecific single chain antibody or an antibody construct having e.g. 90%, 95%, 96%, 97%, 98% or 99% sequence identity to the amino acid sequences of the EpCAMxCD3 bispecific single chain antibody can be detected by methods as illustrated e.g. in WO2005/040220 (PCT/EP2004/011646).

It is also envisaged that the methods of the present invention are further characterized by the, preferably concomitant, administration of a glucocorticoid.

As it is shown in the appended Example 2, glucocorticoids were found to somewhat mitigate the above described increase in liver enzymes in the course of the methods of treatment of the present invention. It is therefore envisaged that the methods of the present invention (and thereby the dosage regimens of the present invention) are further characterized by the optional administration of at least one glucocorticoid. Said administration is preferably concomitant to the first and/or second period of time as defined herein. Glucocorticoids (GC) are a class of steroid hormones that bind to the glucocorticoid receptor (GR), which is present in almost every vertebrate animal cell, including humans. These compounds are potent anti-inflammatory agents, regardless of the inflammation's cause. Glucocorticoids suppress, inter alia, the cell-mediated immunity by inhibiting genes that code for the cytokines IL-1, IL-2, IL-3, IL-4, IL-5, IL-6, IL-8 and IFN-γ.

As used herein, the term “glucocorticoid” comprises at least cortisone, cortisol, cloprednol, prednisone, prednisolone, methylprednisolone, deflazacort, fluocortolone, triamcinolone, dexamethasone, and beatamethasone. Prednisone, prednisolone and/or methylprednisolone are thereby preferred.

EpCAM is a pan-epithelial differentiation antigen that is expressed on the (baso-lateral) cell surface of almost all carcinomas. A carcinoma thereby denotes any malignant cancer that arises from epithelial cells. Carcinomas invade surrounding tissues and organs and may metastasize, or spread, to lymph nodes and other sites. The term “EpCAM positive epithelial cancer cells” as used herein, therefore refers to all kinds of carcinomas, including single or metastatic cells thereof, which express EpCAM on their cell-surface. Examples of EpCAM positive epithelial cancer (cells) are gastrointestinal cancer (cells), lung cancer (cells), prostate cancer (cells) and ovarian cancer (cells). “Gastrointestinal” includes for example the esophagus, stomach (gastric), small and large intestines, bladder, gallbladder, liver and pancreas. The main types of lung cancer are small cell lung carcinoma and non-small cell lung carcinoma.

Cancer cells can break away, leak, or spill from a primary tumor, enter lymphatic and blood vessels, circulate through the bloodstream, and be deposited within normal tissue elsewhere in the body. Most malignant tumors and other malignant neoplasms can metastasize, although in varying degrees. Theses metastasizing cancer cells are sometimes also denoted “metastatic variants” or “metastases”. The metastasizing cancer cells are specifically included in the scope of the present invention.

In a preferred embodiment of the methods of the present invention, said gastrointestinal cancer is gastric cancer, colorectal cancer, or metastatic variants thereof and said lung cancer is small lung cancer, non-small lung cancer (NSCLC), or metastatic variants thereof.

Also preferred are adenocarcinoma of the lung, adenocarcinoma of the gastro-esophageal junction, hormone-refractory prostate cancer, ovarian cancer and breast cancer to name some.

In another preferred embodiment of the methods of the invention, the method is for the treatment, amelioration or elimination of minimal residual disease (MRD) in a patient with (metastatic) EpCAM positive epithelial cancer cells. “MRD” is the name given, to small numbers of cancer cells that remain in the patient during treatment, or after treatment when the patient is in remission (i.e. when the patient shows no symptoms or signs of disease). It is the major cause of relapse in epithelial cancer and leukaemia.

In a further embodiment, the present invention relates to a method for:
(i) administering an EpCAMxCD3 bispecific antibody to a human patient, or
(ii) treating EpCAM positive epithelial cancer cells in a human patient; or
(iii) ameliorating or preventing a medical condition mediated by the administration of an EpCAMxCD3 bispecific antibody to a human patient;
   said method comprising:
(a) administering continually an EpCAMxCD3 bispecific antibody such that the serum level of at least one liver enzyme is increased to grade 4 or less (preferably to grade 3) and subsequently decreased to grade 2; and consecutively
(b) administering said antibody such that it is therapeutically active.

In a further embodiment of the methods of the invention, one treatment cycle (including the first and second period of time, and optionally also the third period of time) is followed by one or more repeated cycle(s) after a treatment-free interval. In a preferred embodiment of the methods of the invention, one treatment cycle (including the first and second period of time, and optionally also the third period of time) is a 4 to 8 week continuous infusion, followed by repeated (a) cycle(s) after a 2 to 6 week treatment-free interval. It is also envisaged that said second treatment cycle differs from the 1^{st} treatment cycle (the “first treatment cycle” denotes in this regard the treatment cycle which is directly prior to the “second treatment cycle”) in that the first period of time and/or dosage of said second cycle is different and/or in that the second period of time and/or dosage is different when compared to the first treatment cycle. Likewise, it is possible that the third period of time (if present in the first treatment cycle) might differ or even be absent from the second cycle (or vice versa. i.e. it might be present in the second cycle and absent from the first cycle). It is also envisaged, that the second treatment cycle merely consists of the second period of time, i.e. it is explicitly envisaged that the second treatment cycle directly starts with a therapeutically active dosage and leaves out the adaptation phase which is characterized by the herein described first dose for a first period of time (including the third period of time if present). Said therapeutically active dosage of the second treatment cycle is either identical to the dose which was used in the second period of time of the first treatment cycle or differs from it (preferably exceeds it). Dosage regimens as depicted in FIG. 8 or exemplified in the appended examples are particularly preferred.

The present invention furthermore relates to an EpCAMxCD3 bispecific antibody for:
(i) administering an EpCAMxCD3 bispecific antibody to a human patient, or
(ii) treating EpCAM positive epithelial cancer cells in a human patient; or
(iii) ameliorating or preventing a medical condition mediated by the administration of an EpCAMxCD3 bispecific antibody to a human patient;
   wherein said antibody is to be administered in accordance with a method or dosage regimen of the present invention.

The present invention also relates to the use of an EpCAMxCD3 bispecific antibody for the preparation/manufacture of a pharmaceutical composition to be used in a method as defined in any one of the preceding claims. The pharmaceutical composition of the present invention may optionally comprise a pharmaceutical carrier. Examples of suitable pharmaceutical carriers are well known in the art and include phosphate buffered saline solutions, sterile solutions etc. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Furthermore, the pharmaceutical composition of the invention may comprise further agents such as glucocorticoids as explained herein elsewhere.

In a further aspect, the present invention relates to a (pharmaceutical) kit or pharmaceutical package comprising the first dose and the second dose as defined herein. Said first and second dose are thereby packaged together in one sealed) pharmaceutical package or kit. It will be understood that the “first dose” and the second dose” encompasses in this regard the respective number of single doses which will be used for a given period of time (either the first or the second period of time). This means for example that the “first dose” or “second dose” which is comprised in the pharmaceutical package or kit of the present invention comprises, for example, 7 daily doses which are separated. The number of packaged daily doses thereby reflects the intended period of time ( ) daily doses if said period of time is X days, Y daily doses if the period of time is Y days and so on). In these embodiments, the (pharmaceutical) kit or pharmaceutical package comprises the daily dosages in separate containers, in a single package. As mentioned herein elsewhere, said separate containers might contain different doses, for example in the context of an increasing dosage during the 1^{st} or 2^{nd} period of time as described herein—the 1^{st} containers might comprise a 3 μg dosage per day (e.g. multiplied by seven) while further containers comprise 12 μg/day (e.g. multiplied by seven)—all these containers, however, still form part of the “first dose”).

Alternatively, it is also envisaged that the intended first dose and/or second dose is not separated into the respective number of daily doses but is contained, either in toto or in part, in one single container (for example an infusion bag), which comprises the required dose for either the first and/or the second period of time either in part (for example for 1 to 3 days) or in toto (i.e. for the first or second period of time). This means that one single container comprises for example 7 daily doses for the “first dose” which is to be used during the first period of time etc.

It will be understood that the (pharmaceutical) kit or pharmaceutical package of the present invention may also comprises more or less daily doses as required for the respective period of time (either separated or not). Alternatively, the (pharmaceutical) kit or pharmaceutical package is prepared such that it contains the required number of daily doses (either separated or not) for the first and second period of time as defined herein, i.e. the “first dose” and the “second dose” in one single package. Such a package is ideally sufficient for one complete treatment of a patient (including the first and the second period of time). Parts of the kit and package of the invention can be packaged individually in vials or bottles or in combination in containers or multicontainer units. The manufacture of the kits follows preferably standard procedures which are known to the person skilled in the art.

Furthermore, the invention relates to a pharmaceutical package or kit comprising the first dose and the second dose as described hereinbefore as active ingredients and written instructions for the sequential use thereof in accordance with the methods of the present invention. Said pharmaceutical package or kit may further comprise a label or imprint indicating that the contents can be used for treating EpCAM positive epithelial cancer cells in a human patient; or for ameliorating or preventing a medical condition mediated by the administration of an EpCAMxCD3 bispecific antibody to a human patient.

It is also envisaged that the pharmaceutical package or kit of the present invention, further comprises means to administer the first and/or the second dose to a patient and/or buffers, vials, teflon bags or infusion bags which are normally used for the infusion of therapeutic agents. “Means” thereby includes one or more article(s) selected from the group consisting of a syringe, a hypodermic needle, a cannula, a catheter, an infusion bag for intravenous administration, intravenous vehicles, vials, buffers, stabilizers, written instructions which aid the skilled person in the preparation of the respective doses and infusions of the invention etc.

It is also envisaged that the pharmaceutical package or kit of the present invention further comprises a glucocorticoid.

In a further aspect, the present invention provides for a pharmaceutical package or kit, wherein said first and/or said second dose is arranged such, that it is suitable for administration/a dosage regimen in accordance with a method of any one of the preceding claims. “Arranged such” includes that the daily doses are packaged apart from each other of together; and/or that the and/or the second dose are packaged in toto, or combinations thereof.

The present invention also relates to the following items:
1. A method (dosage regimen) for administering an EpCAMxCD3 bispecific antibody to a human patient, comprising:
   (a) administering continually a first dose of said antibody for a first period of time; and consecutively
   (b) administering continually a second dose of said antibody for a second period of time;
   wherein said second dose exceeds said first dose.
2. A method for treating EpCAM positive epithelial cancer cells in a human patient, said method comprising:
   (a) administering continually a first dose of an EpCAMxCD3 bispecific antibody for a first period of time; and consecutively
   (b) administering continually a second dose of said antibody for a second period of time;
   wherein said second dose exceeds said first dose.
3. A method for ameliorating and/or preventing a medical condition, preferably an adverse effect, mediated by the continued (therapeutic) administration of an EpCAMxCD3 bispecific antibody to a human patient, said method comprising:
   (a) administering continually a first dose of said antibody for a first period of time, and consecutively
   (b) administering continually a second dose of said antibody for a second period of time;
   wherein said second dose exceeds said first dose.
4. The method of item 1, 2 or 3, wherein said human patient comprises or is assumed to comprise EpCAM positive epithelial cancer cells.
5. The method of any one of the preceding items, wherein the route of administration in step (a) and/or the route of administration in step (b) is intravenous.
6. The method of item 3, wherein said medical condition, preferably said adverse effect, is characterized by an increase of the serum level of at least one liver enzyme.
7. The method of any one of the preceding items, wherein said second period of time exceeds said first period of time.
8. The method of any one of the preceding items, wherein said first period of time is at least 1, 2, 3, 4, 5, 6, 7 days (or more), 7 days being preferred.
9. The method of any one of the preceding items, wherein said first period of time is characterized by an increase of the serum level of at least one liver enzyme up to grade 3 or 4.
10. The method of item 9, wherein said first period of time persists until the increased serum level of said liver enzyme is decreased to grade 2.
11. The method of any one of the preceding items, wherein the first period of time is 4 days, provided that the serum level of said at least one liver enzyme is grade 2 or below.
12. The method of any one of items 6 to 11, wherein said at least one liver enzyme is AST and/or ALT and optionally also GGT and/or AP.
13. The method of any one of the preceding items, wherein said second period of time persists at least until the CD8+-T-cells of said patient are activated.
14. The method of item 13, wherein said activation is characterized by a CD25 and/or CD69-positive phenotype of at least 20% of said CD8+-T-cells.
15. The method of any one of the preceding items, wherein said second period of time is at least 2, 3, 4, 5 or 6 weeks, 3 weeks being preferred.
16. The method of any one of the preceding items, wherein said second period of time is at least 19 days.
17. The method of any one of the preceding items, wherein said first period of time is between 1 and 10 days, and that second period of time is at least 19 days.
18 The method of item 17, wherein said first period of time is 7 to 9 days and that second period of time is 19 to 21 days
19. The method of item 18, wherein said first period of time is 7 days and that second period of time is 21 days.
20. The method of any one of the preceding items, wherein said second dose is therapeutically active.
21. The method of item 20, wherein said therapeutic activity of said second dose is characterized by activated CD8+-T-cells.
22. The method of item 21, wherein said activation is characterized by a CD25 and/or CD69-positive phenotype of at least 20% of said CD8+-T-cells (in relation to the CD25 and/or CD69-positive phenotype prior to the second period).
23. The method of any one of the preceding items, wherein said first dose is such that the serum level of at least one liver enzyme increases to a serum level of grade 3 or 4 and decreases again to a serum level of grade 2 within the first period of time.
24. The method of item 23 wherein said at least one liver enzyme is AST and/or ALT and optionally also GGT and/or AP.
25. The method of any one of the preceding items, wherein said first dose is between 1 and 6 μg/d, 1 to 3 μg/d being preferred.
26. The method of any one of the preceding items, wherein said second dose is between 10 and 120 μg/d (or more, if required under therapeutically relevant aspects).
27. The method of any one of the preceding items, wherein said first dose is 1 to 3 μg/d and that second dose is 20 to 90 μg/d.
28. The method of any one of the preceding items, wherein said bispecific antibody is a single chain antibody.
29. The method of any one of the preceding items, wherein said antibody is MT110.
30. The method of any one of the preceding items, further characterized by the (concomitant) administration of a glucocorticoid.
31. The method of item 30, wherein said glucocorticoid is prednisone, prednisolone and/or methylprednisolone.
32. The method of item 1 or 4, wherein said EpCAM positive epithelial cancer cells are gastrointestinal and/or lung cancer cells.
33. The method of item 32, wherein said gastrointestinal cancer is gastric cancer, colorectal cancer, or metastatic variants thereof and said lung cancer is small lung cancer, non-small lung cancer, or metastatic variants thereof.

### FIGURES

The figures show:

FIG. 1 T cell activating potency of the EpCAMxCD3 bispecific single chain antibody in accordance with Example 1

FIG. 2 Exemplary dosage regimen in accordance with Example 2

FIG. 3 Generation of MT110 and Mode of Action

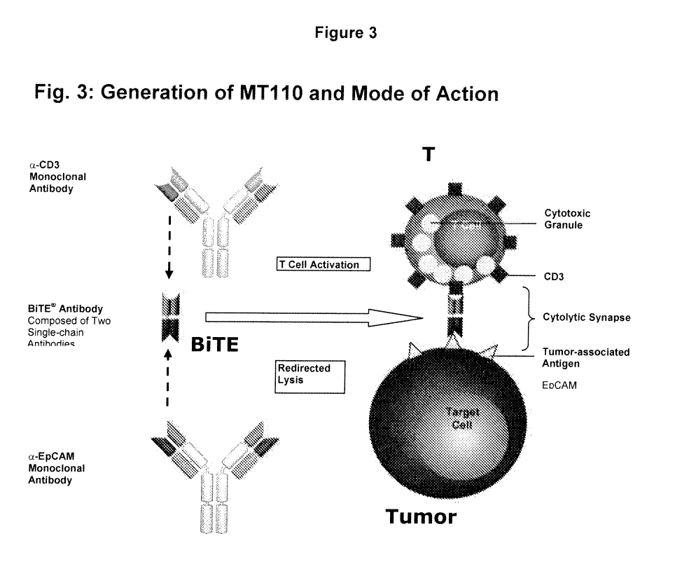

FIG. 4 MT110 plasma levels for patients treated with MT110 maintenance doses of 10 or 12 μg/day in schedule A, B or C show a comparable profile

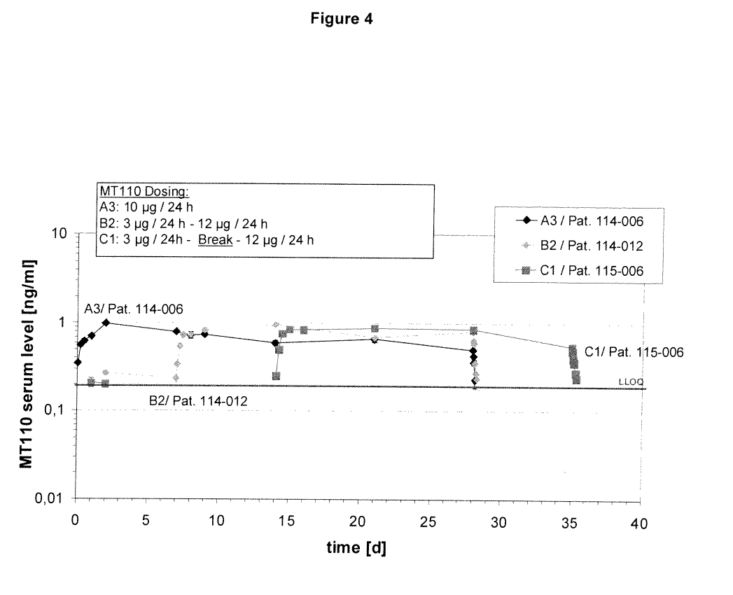

FIG. 5A Lymphocytes re-distribution observed after start of MT110 infusion and after dose escalation on day 7 in patient 114-012 (cohort B2: 3→12 μg/d)

FIG. 5B T cell activation observed after start of MT110 infusion and after dose escalation on day 7 in patient 114-012 (cohort B2: 3→12 μg/d)

FIG. 6A Resected lung lesion. Large number of CD3 positive lymphocytes (red arrows), necrotic tissue (green arrow), tumor cells (orange arrow).

FIG. 6B Resected lung lesion. Infiltration of CD8 positive lymphocytes (red arrow).

FIG. 7 Liver parenchyma at peak of transaminase

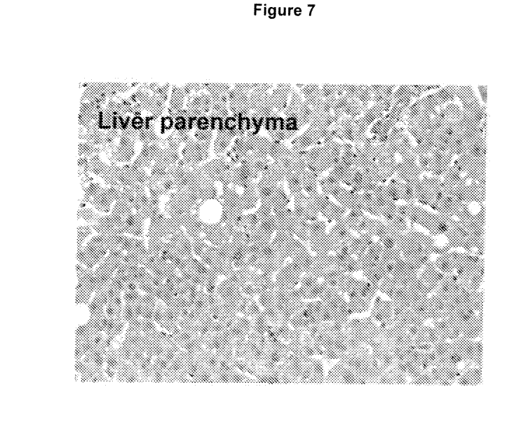

FIG. 8 Specific dosage regime of the present invention

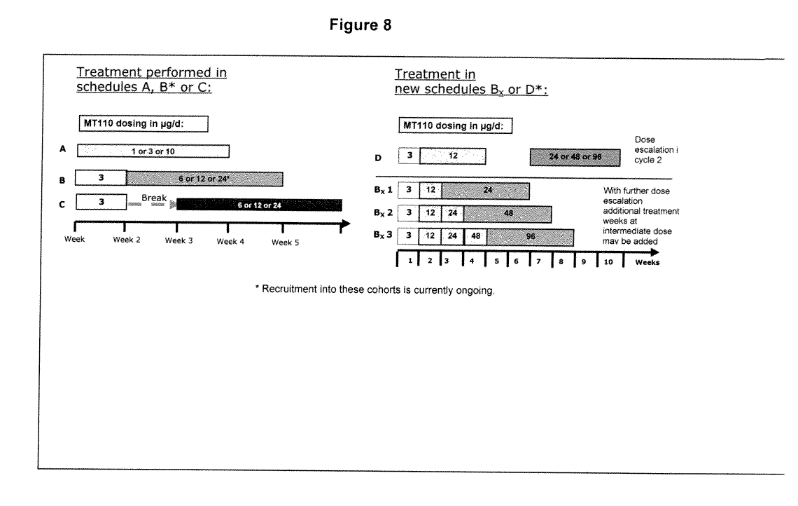

### EXAMPLES

The following examples illustrate the invention. These examples should not be construed as to limit the scope of this invention. The examples are included for purposes of illustration and the present invention is limited only by the claims.

### Example 1

EpCAMxCD3 bispecific single chain antibody mediates T cell specific cytotoxicity to EpCAM positive target cells and thereby activation of T cells measurable by release of cytokines and upregulation of T cell surface markers. This process is strictly dose-dependent and relies completely on the presence of EpCAM positive target cells. The stringent biological effects of the EpCAMxCD3 bispecific single chain antibody MT110 on human T cells were analyzed in vitro as follows:

Human PBMC of random healthy donors were incubated with the indicated concentrations of the EpCAMxCD3 bispecific single chain antibody in the absence or presence of 0.2%, 2% or 10% KatoIII cells, naturally expressing EpCAM. After incubation for 40 hours at 37° C. in a humidified incubator cells were stained with fluorescence-labeled antibodies against CD4. CD8 and CD25 to identify effector cells (CD4 or CD8-positive) and their activation status (de novo expression of CD25) by flow cytometry. The percentage of CD25⁺ T cells was plotted against the logarithm of BiTE concentration using Prism software (Graph Pad Software Inc., version 4.02). The resulting dose-response curves were analyzed with the integrated four-parameter nonlinear fit model also calculating concentrations of the half-maximal kill (EC₅₀) as indicator for specific bioactivity. The supernatants of samples containing 100 ng/ml of the EpCAMxCD3 bispecific single chain antibody were analyzed for cytokine contents using the human Th1/Th2 cytometric bead array (CBA) kit (BD Bioscience) according to manufacturer's instructions.

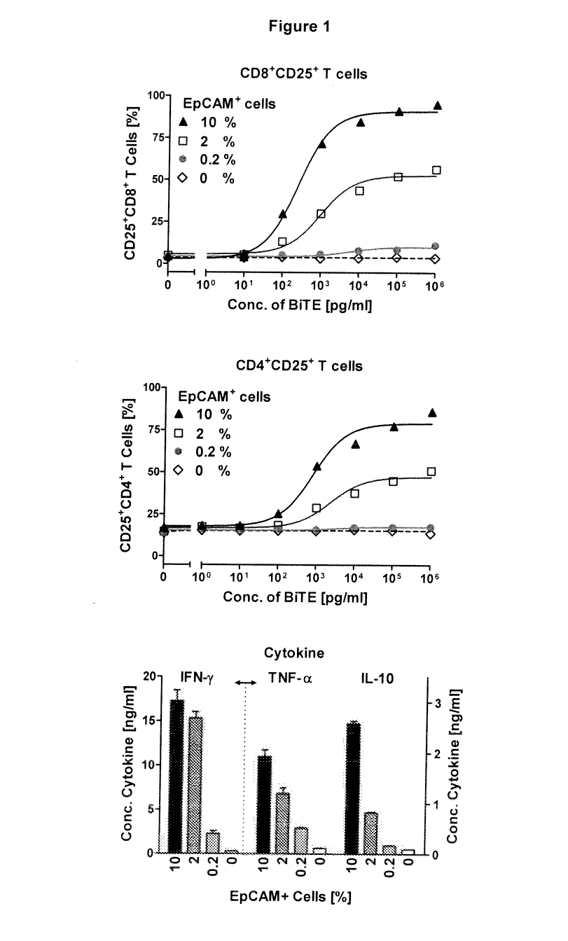

The T cell activating potency of the EpCAMxCD3 bispecific single chain antibody is shown in FIG. 1. Addition of 10% EpCAM⁺ cells to human PBMC led to activation of approximately 95% of CD8⁺ and 85% of CD4⁺ T cells in an antibody dose-dependent manner within 40 hours. Addition of 2% EpCAM⁺ cells resulted in an intermediate activation of app. 50% of CD8⁺ and 25% of CD4⁺ T cells whereas addition of only 0.2% EpCAM⁺ cells hardly induced CD25 expression on the effector cells for any concentration of the BiTE tested. The EC50 values increased with decreasing amount of target cells present and were for 10, 2 and 0.2% KatoIII cells present 0.26, 0.99, and 5.98 ng/ml for CD8⁺ T lymphocytes and 0.8, 2.35, and 3.50 ng/ml for CD4⁺ T lymphocytes, respectively. The BiTE-mediated upregulation of CD25 was accompanied by release of IFN-γ, TNF-α, and IL-10 from human PBMC, which strength correlated well with the percentage of EpCAM positive target cells present. No cytokine release or upregulation of CD25 on CD8⁺ or CD4⁺ T cell was detected in the absence of EpCAM⁺ target cells for any BiTE concentration evaluated. This might be indicative for high potency of the EpCAMxCD3 bispecific single chain antibody in an EpCAM-rich tumor environment and concurrent good tolerability in tissues where EpCAM is not directly accessible to T cells.

### Example 2

### Safety and Pharmacology of the EpCAM/CD3-Bispecific BiTE Antibody MT110 in Patients with Metastatic Colorectal, Gastric or Lung Cancer

### 1.1 Study Design

Primary Objectives: To assess safety and tolerability
Secondary Objectives: To assess pharmacokinetics (PK), pharmacodynamics (PD) and anti-tumor activity of MT110
Patients: Locally advanced, recurrent or metastatic solid tumors known to widely express EpCAM
   Adenocarcinoma of the lung
   Small cell lung cancer
   Gastric cancer
   Colorectal cancer

### Other Key Eligibility Criteria:

Standard therapeutic options are exhausted or declined
At least one course of previous chemotherapy
ECOG performance status 2
Ability to understand the patient information and informed consent form
No evidence of CNS metastases on baseline CT or MRI scan or other history of CNS pathology
Neutrophil count <1,500/mm³ (=1.5×109/l)
Platelet count <100,000/mm³ (=100×109/l)
WBC<3×109/l; hemoglobin <9.0 g/dl
No abnormal renal or hepatic function
No O2 saturation of <92% (under room air condition)
No concurrent anti-neoplastic therapy, except palliative radiotherapy
No presence of human anti-murine antibodies (HAMA)

### 1.2 MT110 Treatment and Dose Escalation

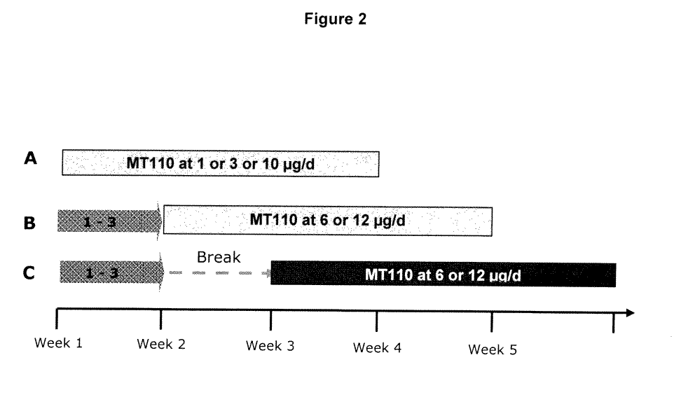

MT110: Continuous intravenous infusion d1-28 Repeated cycles until disease progression with 2 to 4 weeks treatment break.
Dose cohorts: MT110 at 1/3/6/10/12/24 (currently ongoing) μg per day, further dose escalation in cohorts B/C planned (see FIG. 2).
   3+3 design depending on occurrence of DLTs. Decision on dose escalation after completion of the first cycle of each patient in the respective cohort.
   Treatment performed with schedule A, B or C.
DLT criteria: Any grade 3 or 4 related AE that persists longer than pre-defined despite adequate patient management-
Concomitant Medication: Prior to MT110 infusion or dose escalation corticosteroid, antihistamine, antacid-
   Further supportive medication permitted-

### 1.3 Assessments

Safety: Continuous assessment of safety parameters (adverse event (AE) reporting according to CTCAE version 3.0), laboratory parameters at least twice daily on days 1 and 2, once daily on days 3 and 5 in weeks 1 and 2; and twice weekly afterwards.
Anti-Tumor Activity: Assessment after each treatment cycle (according to RECIST version 1.0 in case of measurable lesions).
PK/PD: Samples for analysis of PK, cytokines, lymphocytes subpopulations, circulating tumor cells and immunogenicity are collected at pre-defined time points
   EpCAM expression is analyzed in paraffin-embedded tumor tissue

### 2. Results

### 2.1 Patients and MT110 Treatment

The data presented herein are as of Sep. 10, 2009, the study is ongoing.
22 patients have started MT110 therapy, the interim analysis includes:
   Demografic data of 20 patients
   Safety and pharmacodynamic data of 20 patients

TABLE 1
Patient demographics per dose cohort

| | MT110 dose cohorts | | | | | | |
|---|---|---|---|---|---|---|---|
| | A1: | A2: | A3: | B1: | B2: | C1: 3break/ | |
| | 1 μg/d | 3 μg/d | 10 μg/d | 3/6 μg/d | 3/12 μg/d | 12 μg/d | Total |
| | N = 6 | N = 3 | N = 2 | N = 4 | N = 4 | N = 1 | N = 20 |
| Characteristic | | | | | | | |
| Median age (years, range) | 64 | 56 | 54 | 62 | 65 | 75 | 63 (44-85) |
| Gender male (n; %) | 4 | 2 | 1 | 4 | 3 | 0 | 12 (67.0%) |
| Diagnosis | | | | | | | |
| CRC (n; %) | 5 | 2 | 1 | 2 | 3 | 1 | 14 (70.0%) |
| Gastric cancer (n; %) | 1 | 1 | 0 | 1 | 0 | 0 | 3 (15.0%) |
| NSCLC (n; %) | 0 | 0 | 1 | 0 | 1 | 0 | 2 (10.0%) |
| SCLC (n; %) | 0 | 0 | 0 | 1 | 0 | 0 | 1 (5.0%) |
| Prior lines of chemo (n; %) | | | | | | | |
| ≧3 | 5 | 2 | 1 | 2 | 3 | 1 | 14 (70.0%) |
| <3 | 1 | 1 | 1 | 2 | 1 | 0 | 6 (30.0%) |
| Prior radiation (n; %) | 1 | 2 | 0 | 2 | 1 | 0 | 6 (30.0%) |
| Prior surgery (n; %) | 6 | 2 | 1 | 4 | 4 | 1 | 18 (90.0%) |
| Liver metastases (n; %) | 3 | 2 | 0 | 3 | 4 | 1 | 13 (65.0%) |
| ECOG 0/1/2 | 1/5/0 | 2/0/1 | 1/1/0 | 2/2/0 | 1/3/0 | 0/0/1 | 7/11/2 |
| Abnormal liver parameters at | 3 | 3 | 1 | 2 | 3 | 1 | 13 (65.0%) |
| baseline | | | | | | | |

### 2.2 Safety & Tolerability

### Adverse Events Summary

Out of 20 patients 17 were able to complete at least one cycle of 4 weeks MT110 intravenous infusion.
Most of the observed clinical adverse events were related to the underlying disease.
Non-hematological clinical adverse events related to MT110 consisted of mild pyrexia and fatigue in few patients, pyrexia was not associated with a first infusion reaction.
Laboratory changes that were related to MT110, occurred primarily in the first week of infusion or after dose escalation, were of short duration and resolved during the course of infusion in most cases. Changes in liver parameters were also transient and asymptomatic; and were not accompanied by pathological findings in imaging, substantial tissue damage or impaired synthesis parameters of the liver.

TABLE 2
Incidence of AEs regardless of relationship
occurring in ≧3 patients

| | Total, N = 20 | | | |
|---|---|---|---|---|
| | Grade 1/2 | | Grade 3/4 | |
| | Pat. | (%) | Pat. | (%) |
| CLINICAL ADVERSE EVENTS | | | | |
| Abdominal pain | 5 | (25.0%) | 0 | (0.0%) |
| Pyrexia | 8 | (40.0%) | 1 | (5.0%) |
| Vomiting | 6 | (30.0%) | 0 | (0.0%) |
| Nasopharyngitis | 5 | (25.0%) | 0 | (0.0%) |
| Cough | 4 | (20.0%) | 0 | (0.0%) |
| Diarrhea | 4 | (20.0%) | 0 | (0.0%) |
| Edema peripheral | 4 | (20.0%) | 0 | (0.0%) |
| Fatigue | 3 | (15.0%) | 0 | (0,0%) |
| LABORATORY CHANGES | | | | |
| Lymphocyte count decreased/ | 2 | (10.0%) | 14 | (70.0%) |
| lymphopenia | | | | |
| Hemoglobin decreased | 2 | (10%) | 1 | (5%) |
| Gamma-glutamyltransferase | 3 | (15.0%) | 13 | (65.0%) |
| increased | | | | |
| Aminotransferases increased | 2 | (10.0%) | 9 | (50.0%) |
| Blood glucose increased/ | 10 | (50.0%) | 7 | (35.0%) |
| hyperglycemia | | | | |
| Blood amylase increased | 2 | (10.0%) | 3 | (15.0%) |
| Lipase increased | 1 | (5.0%) | 5 | (25.0%) |
| C-reactive protein increased | 4 | (20.0%) | 0 | (0.0%) |
| Blood alkaline phosphatase | 2 | (10.0%) | 1 | (5.0%) |
| increased | | | | |
| Blood bilirubin increased | 4 | (20.0%) | 0 | (0.0%) |
| Hypocalcemia | 3 | (15%) | 0 | (0.0%) |
| Blood lactate dehydrogenase | 3 | (15.0%) | 1 | (5.0%) |
| increased | | | | |

TABLE 3
Incidence of adverse events related to MT110 occurring in >1 patient

| | A1 (n = 6) | | A2 (n = 3) | | A3 (n = 2) | | B1 (n = 4) | | B2 (n = 4) | |
|---|---|---|---|---|---|---|---|---|---|---|
| | G1/2 | G3/4 | G1/2 | G3/4 | G1/2 | G3/4 | G1/2 | G3/4 | G1/2 | G3/4 |
| CLINICAL ADVERSE EVENTS | | | | | | | | | | |
| Pyrexia | 2 | 0 | 1 | 0 | 0 | 0 | 1 | 0 | 0 | 0 |
| Fatigue | 2 | 0 | 0 | 0 | 0 | 0 | 0 | 0 | 0 | 0 |
| LABORATORY CHANGES* | | | | | | | | | | |
| Lymphopenia/lymphocyte count | 1 | 4 | 0 | 3 | 0 | 2 | 0 | 4 | 1 | 0 |
| decreased | | | | | | | | | | |
| Gamma-glutamyltransferase increased | 2 | 3 | 0 | 2 | 0 | 2 | 1 | 2 | 0 | 3 |
| Aminotransferases increased | 2 | 2 | 0 | 2 | 0 | 2 | 0 | 3 | 0 | 1 |
| Blood amylase increased | 0 | 0 | 1 | 0 | 1 | 0 | 0 | 1 | 0 | 1 |
| Lipase increased | 1 | 0 | 0 | 1 | 0 | 1 | 0 | 1 | 0 | 1 |
| Blood alkaline phosphatase increased | 1 | 1 | 0 | 0 | 0 | 0 | 0 | 0 | 1 | 0 |
| Blood bilirubin increased | 2 | 0 | 0 | 0 | 0 | 0 | 0 | 0 | 1 | 0 |
| Blood lactate dehydrogenase increased | 1 | 0 | 0 | 1 | 0 | 0 | 1 | 0 | 0 | 0 |
| Hypocalcemia | 0 | 0 | 0 | 0 | 0 | 0 | 0 | 0 | 2 | 0 |

TABLE 4
Reported dose-limiting toxicities

| | MT110 | | | | |
|---|---|---|---|---|---|
| Patient | dose | Tumor | Event | Action | Outcome |
| 114-002 | 1 μg/d | Gastric | Grade 4 | Corticosteroid | Resolved |
| (A1) | | cancer | increase in | administration | |
| | | | AST + ALT | | |
| 114-007 | 10 μg/d | NSCLC | Grade 3 ALT | MT110 | Resolved |
| (A3) | | | for >72 hrs | infusion | |
| | | | | discontinued | |

### 2.3 Pharmacokinetic Profile

### Summary of PK Parameters

The serum halflife for MT110 was determined with 3.5-6.8 hrs.
After normalization for dose and body weight a dose linearity can be assumed for the dose levels tested to date.

### 2.4 Pharmacodynamic Markers

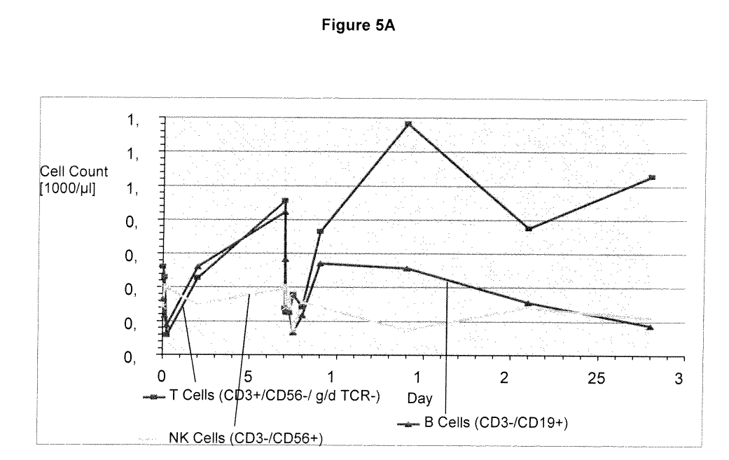

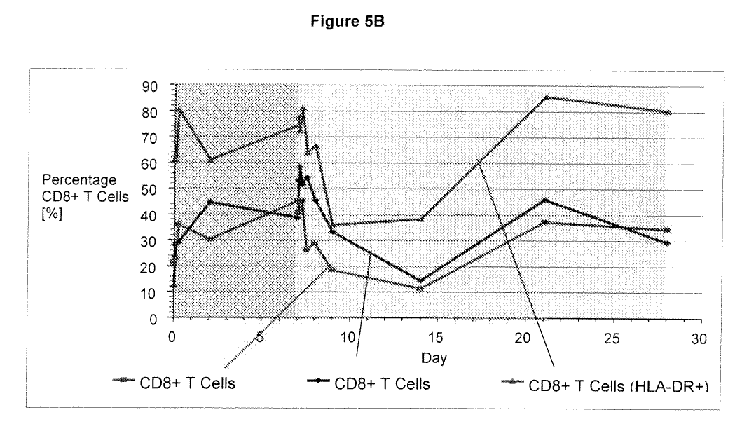

None of the patients showed a significant systemic cytokine release, low IL-6 levels were measurable at different time points and single measurable levels of IFNγ and IL-10 were seen in some patients
Re-distribution of lymphocytes was observed after start of MT110 infusion as well as after dose escalation (FIG. 5a) in all patients
First signs of T cell activation were observed in patients with clinical benefit after first cycle (FIG. 5b)
First analysis of circulating tumor cells via CellSearch method revealed up to 6 cells per 7.5 ml sample from CRC patients

### 2.5 Outcome of Patients

### Tumor Assessment According to RECIST Criteria

Disease stabilization was observed in 7 of 18 evaluable patients after first cycle with a median duration of 91 days (range 29+ to 150 days)

### Case Report: Evidence of Biological Antitumor Activity

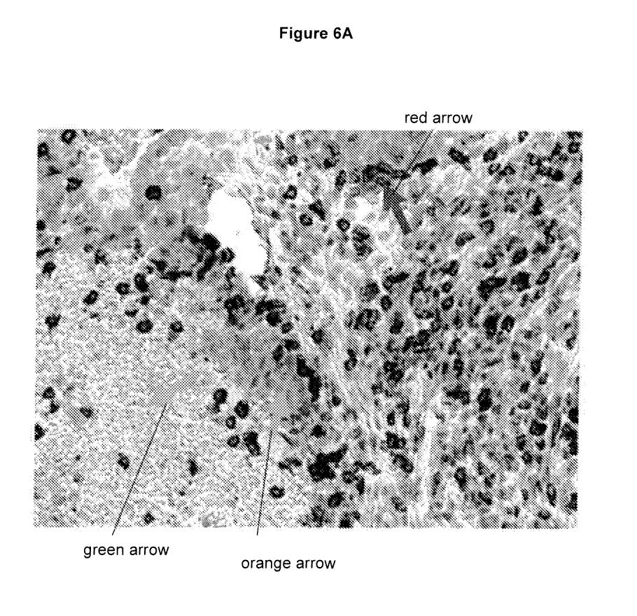

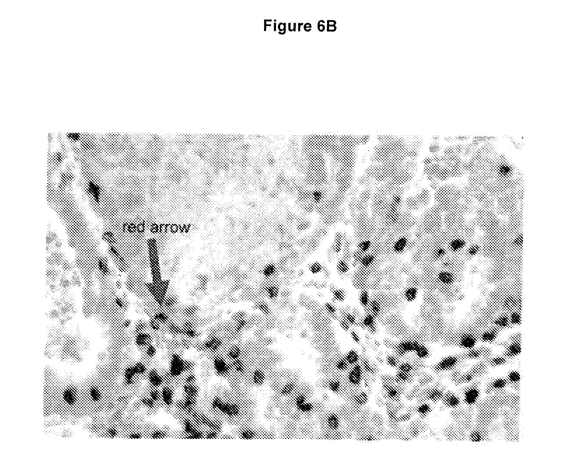

Patient (female, 85 years) presented metastatic lesions in the lung of a CRC at study entry
Treatment with MT110 of 1 μg/d for 28 days
Patient underwent surgical resection of a lung lesion 80 days after start of MT110, pathology revealed:
   >70% of necrotic tissue in this lesion (FIG. 6A)
   High number of infiltrating T-lymphocytes including CD8 positive cells in and around tumor tissue (FIG. 6B)

### 3. Summary

Twenty eligible patients were treated in five dose cohorts and received a total of twenty five MT110-cycles up to date:
MT110 intravenous infusion over 28 days is clinically very well tolerated:
   Mild pyrexia and fatigue occurred in few patients and were related to the study medication.
   No signs of relevant systemic cytokine release was observed.
   Besides initial transient lymphopenia, a transient asymptomatic increase in liver enzymes up to Grade 3/4, was the most frequent laboratory abnormality.
First signs of biological activity
   MT110 caused a rapid redistribution of lymphocytes shortly after start of infusion. Signs of T cell expansion/-activation were seen in patients with clinical benefit after 4 weeks.
   Disease stabilization according to RECIST was confirmed in 7 of 18 patients, lasting 91 days in median.
   In one patient, a lung metastasis was resected 11 weeks after start of MT110 treatment. Immunohistochemistry revealed tumor cell necrosis and a massive T cell infiltration as possible evidence of MT110 activity.
None of the patients developed antibodies against MT110.

### 4. Conclusion

MT110 can be safely administered intravenously to patients with advanced EpCAM-expressing solid tumors.
First signs of biological activity have been observed at clinically well tolerated doses.
Evaluation of BiTE antibody MT110 at escalating doses is currently ongoing.

### Example 3

### Comparison of Changes in Liver Parameters in the First Patient Cohorts Treated with MT110 in The study MT110-101

In the following tables the increase of liver parameters, presented by the mean amplitudes of peak blood level (Table 1) and mean blood level values (Table 2), is analyzed for the respective patient group in dependence of the MT110 dose, treatment schedule and corticosteroid therapy. The aminotransferases AST and ALT were the liver parameters which were mainly affected by the MT110 infusion.

Amplitudes of peak blood levels of the respective liver parameter were calculated by subtracting the baseline value measured at the screening assessment from the peak level measured in the first week of MT110 infusion at the respective dose for each patient in this group. The resulting amplitude values for each patient of a group were used to calculate the mean for the respective group.

Amplitudes of mean blood values were calculated by subtracting the baseline value measured at the screening assessment from the mean level calculated from all measurement values of the first week of MT110 infusion at the respective dose for each patient in this group. The resulting amplitude values for each patient of a group were used to calculate the mean for the respective group.

The comparison of the different groups can be summarized as follows:
The additional concomitant corticosteroid therapy can reduce the increase in liver parameters (mainly AST and ALT) for patients treated with MT110 at 1 μg/d (group II compared to group I).
With increasing MT110 dose the peak and mean blood levels of liver parameters show increasing values despite the concomitant corticosteroid therapy (group V and III compared to group II).
   Conclusion: The increase in liver parameters can not be prevented by concomitant corticosteroid therapy.
A further increase of the corticosteroid dose can also not prevent the increase in liver parameters with increasing dose (group IV compared to group III).
Start of the MT110 treatment at a low dose in the first week and at a higher dose starting week two results in an only mild increase in liver enzymes in the second week (group VII compared to group V).
   Conclusion: Further dose escalation is possible with a low MT110 starting dose for the first week of the infusion.

TABLE 5
Mean of amplitudes between peak and screening values of first
treatment week at given dose without standard deviation

| | AST | ALT | GGT | AP | Bili |
|---|---|---|---|---|---|
| | (U/l) | (U/l) | (U/l) | (U/l) | (mg/dl) |
| | peak | peak | peak | peak | peak |
| | w 1 | w 1 | w 1 | w 1 | w 1 |
| | above | above | above | above | above |
| MT110 Dose | Scr | Scr | Scr | Scr | Scr |
| Cohort | value | value | value | value | value |
| I: 1 μg + | 438.0 | 436.7 | 276.3 | 252.3 | 0.6 |
| 1 × 100 mg | | | | | |
| d 0 cort. | | | | | |
| II: 1 μg + | 7.8 | 24.3 | 79.3 | 30.3 | 0.2 |
| 2-3 × 100 mg | | | | | |
| cort. d 0-2 | | | | | |
| III: 3 μg + | 161.4 | 152.1 | 209.4 | 36.3 | 0.3 |
| 2-3 × 100 mg | | | | | |
| cort. d 0-2 | | | | | |
| IV: 3 μg + | 212.0 | 272.8 | 152.8 | 12.3 | 0.1 |
| 1 g + 2 × 500 mg | | | | | |
| cort. d 0-2 | | | | | |
| V: 10 μg + | 540 | 510 | 627.5 | 136.5 | 0.6 |
| 2-3 × 100 mg | | | | | |
| cort. d 0-2 | | | | | |
| IV: 6 μg + | 69.0 | 175.3 | 234.0 | 21.3 | 0.4 |
| 1 g + 2 × 500 mg | | | | | |
| cort. d 0-2 after | | | | | |
| 1 wk low dose (3 μg) | | | | | |
| VII: 12 μg + | 94.7 | 155.7 | 378.7 | 60.0 | 0.7 |
| 2-3 × 100 mg | | | | | |
| cort. d 0-2 after | | | | | |
| 1 wk low dose (3 μg) | | | | | |

TABLE 6
Mean of amplitudes between mean and screening values of first
treatment week at given dose without standard deviation

| | AST | ALT | GGT | AP | Bili |
|---|---|---|---|---|---|
| | (U/l) | (U/l) | (U/l) | (U/l) | (mg/dl) |
| | mean | mean | mean | mean | mean |
| | w 1 | w 1 | w 1 | w 1 | w 1 |
| | above | above | above | above | above |
| MT110 Dose | Scr | Scr | Scr | Scr | Scr |
| Cohort | value | value | value | value | value |
| I: 1 μg + | 132.2 | 235.4 | 185.4 | 160.2 | 0.3 |
| 1 × 100 mg | | | | | |
| d 0 cort. | | | | | |
| II: 1 μg + | 18.6 | 9.2 | 120.6 | 9.4 | 0.0 |
| 2-3 × 100 mg | | | | | |
| cort. d 0-2 | | | | | |
| III: 3 μg + | 50.5 | 70.1 | 113.4 | 14.5 | 0.1 |
| 2-3 × 100 mg | | | | | |
| cort. d 0-2 | | | | | |
| IV: 3 μg + | 64.6 | 120.1 | 72.1 | 2.0 | 0.0 |
| 1 g + 2 × 500 mg | | | | | |
| cort. d 0-2 | | | | | |
| V: 10 μg + | 161.7 | 277.3 | 322.5 | 87.9 | 0.1 |
| 2-3 × 100 mg | | | | | |
| cort. d 0-2 | | | | | |
| IV: 6 μg + | 13.0 | 105.2 | 163.1 | 0.0 | 0.2 |
| 1 g + 2 × 500 mg | | | | | |
| cort. d 0-2 after | | | | | |
| 1 wk low dose (3 μg) | | | | | |
| VII: 12 μg + | 26.2 | 96.5 | 220.8 | 23.2 | 0.2 |
| 2-3 × 100 mg | | | | | |
| cort. d 0-2 after | | | | | |
| 1 wk low dose (3 μg) | | | | | |

Wk: week
W 1: week 1
Scr: screening assessment
Cort: corticosteroid

### Example 4

TABLE 8

| | Dose Cohorts | | | | | | | | | |
|---|---|---|---|---|---|---|---|---|---|---|
| | | | | B1: | B2: | B3: | C1: 3/ | C2: 3/ | D1: Cyc 1 = | |
| | A1: | A2: | A3: | 3/6 | 3/12 | 3/24 | Break/ | Break/ | 3/12 Cyc 2 = | |
| | 1 μg/d | 3 μg/d | 10 μg/d | μg/d | μg/d | μg/d | 12 μg/d | 24 μg/d | 24 μg/d | Total |
| | N = 6 | N = 3 | N = 2* | N = 4+ | N = 4+ | N = 2++ | N = 4+ | N = 3 | N = 1++ | N = 29 |
| Characteristic | | | | | | | | | | |
| Median Age (years, Range) | 64 | 56 | 54 | 62 | 65 | 55 | 64 | 69 | 58 | 64 (37-85) |
| Gender Male (n, %) | 4 | 2 | 1 | 4 | 3 | 1 | 2 | 3 | 0 | 20 (69%) |
| ECOG 0/1/2 | 1/5/0 | 2/0/1 | 1/1/0 | 2/2/0 | 1/3/0 | 0/1/1 | 0/2/2 | 2/1/0 | 0/1/0 | 9/16/4 |
| Diagnosis | | | | | | | | | | |
| CRC (n, %) | 5 | 2 | 1 | 2 | 3 | 1 | 3 | 3 | 1 | 21 (72%) |
| Gastric (n, %) | 1 | 1 | 0 | 1 | 0 | 1 | 1 | 0 | 0 | 5 (17%) |
| NSCLC (n, %) | 0 | 0 | 1 | 0 | 1 | 0 | 0 | 0 | 0 | 2 (7%) |
| SCLC (n, %) | 0 | 0 | 0 | 1 | 0 | 0 | 0 | 0 | 0 | 1 (3%) |
| Prior lines of chemo (n, %) | | | | | | | | | | |
| ≧3 | 5 | 2 | 1 | 2 | 3 | 2 | 2 | 1 | 0 | 18 (62%) |
| <3 | 1 | 1 | 1 | 2 | 1 | 0 | 2 | 2 | 1 | 11 (38%) |
| Prior radiation (n, %) | 1 | 2 | 0 | 2 | 1 | 1 | 1 | 2 | 1 | 11 (38%) |
| Prior surgery (n, %) | 6 | 2 | 1 | 4 | 4 | 2 | 3 | 3 | 1 | 26 (90%) |
| Liver metastases (n, %) | 3 | 2 | 0 | 3 | 4 | 1 | 4 | 2 | 1 | 20 (69%) |
| Abnormal liver parameters at | 3 | 3 | 1 | 2 | 4 | 1 | 3 | 1 | 1 | 19 (66%) |
| baseline | | | | | | | | | | |

*Monitoring committee decision to open cohort B1 instead of finishing recruitment in A3
++Ongoing cohort
+One patient in cohort replaced

TABLE 9

| | A1 | | A2 | | A3 | | B1 | | B2 | | B3* | | C1 | | C2 | |
|---|---|---|---|---|---|---|---|---|---|---|---|---|---|---|---|---|
| | (n = 6) | | (n = 3) | | (n = 2) | | (n = 4) | | (n = 4) | | (n = 2) | | (n = 4) | | (n = 3) | |
| | DOSE-LIMITING TOXICITIES | | | | | | | | | | | | | | | |
| | 1 | | | | 1 | | | | | | 1 | | | | 2 | |
| | G1/2 | G3/4 | G1/2 | G3/4 | G1/2 | G3/4 | G1/2 | G3/4 | G1/2 | G3/4 | G1/2 | G3/4 | G1/2 | G3/4 | G1/2 | G3/4 |
| CLINICAL | | | | | | | | | | | | | | | | |
| ADVERSE EVENTS | | | | | | | | | | | | | | | | |
| Pyrexia | 1 | 0 | 1 | 0 | 0 | 0 | 1 | 0 | 0 | 0 | 1 | 0 | 3 | 0 | 2 | 0 |
| Nausea | 0 | 0 | 0 | 0 | 0 | 0 | 0 | 0 | 0 | 0 | 0 | 0 | 3 | 0 | 2 | 0 |
| Vomiting | 0 | 0 | 0 | 0 | 1 | 0 | 0 | 0 | 0 | 0 | 0 | 0 | 2 | 0 | 2 | 0 |
| Diarrhea | 0 | 0 | 0 | 0 | 0 | 0 | 0 | 0 | 0 | 0 | 0 | 0 | 2 | 0 | 0 | 1Δ |
| Fatigue | 2 | 0 | 0 | 0 | 0 | 0 | 0 | 0 | 0 | 0 | 0 | 0 | 0 | 0 | 1 | 0 |
| LABORATORY | | | | | | | | | | | | | | | | |
| CHANGES | | | | | | | | | | | | | | | | |
| Lymphocyte count | 1 | 4 | 0 | 3 | 0 | 2 | 0 | 4 | 1 | 1 | 0 | 1 | 0 | 4 | 0 | 3 |
| decreased/ | | | | | | | | | | | | | | | | |
| Lymphopenia | | | | | | | | | | | | | | | | |
| Gamma- | 2 | 3 | 0 | 2 | 0 | 2 | 1 | 2 | 0 | 3 | 0 | 2 | 0 | 4 | 0 | 3 |
| glutamyltransfer- | | | | | | | | | | | | | | | | |
| ase increased | | | | | | | | | | | | | | | | |
| Aminotransfer- | 2 | 2Δ | 0 | 2 | 0 | 2Δ | 0 | 3 | 0 | 1 | 0 | 2Δ | 1 | 3 | 1 | 2 |
| ases increased | | | | | | | | | | | | | | | | |
| Lipase increased | 1 | 1 | 0 | 1 | 0 | 1 | 1 | 1 | 0 | 1 | 0 | 1 | 0 | 2 | 2 | 1 |
| Blood bilirubin | 2 | 0 | 0 | 0 | 0 | 0 | 0 | 1 | 0 | 0 | 1 | 1 | 2 | 0 | 2 | 1Δ |
| increased | | | | | | | | | | | | | | | | |
| Blood amylase | 0 | 1 | 1 | 0 | 1 | 0 | 0 | 1 | 0 | 1 | 1 | 0 | 0 | 0 | 1 | 1 |
| increased | | | | | | | | | | | | | | | | |
| Blood alkaline | 1 | 1 | 0 | 0 | 0 | 0 | 0 | 0 | 1 | 0 | 0 | 0 | 1 | 0 | 0 | 1 |
| phosphatase | | | | | | | | | | | | | | | | |
| increased | | | | | | | | | | | | | | | | |
| Blood lactate | 1 | 0 | 0 | 1 | 0 | 0 | 1 | 0 | 0 | 0 | 0 | 0 | 0 | 0 | 1 | 1 |
| dehydrogenase | | | | | | | | | | | | | | | | |
| increased | | | | | | | | | | | | | | | | |
| Glutamate | 0 | 1 | 0 | 0 | 0 | 0 | 0 | 1 | 0 | 0 | 0 | 0 | 0 | 1 | 0 | 2 |
| dehydrogenase | | | | | | | | | | | | | | | | |
| increased | | | | | | | | | | | | | | | | |
| Blood glucose | 0 | 0 | 0 | 0 | 0 | 0 | 0 | 0 | 1 | 0 | 0 | 0 | 0 | 1 | 1 | 0 |
| increased/ | | | | | | | | | | | | | | | | |
| Hyperglycaemia | | | | | | | | | | | | | | | | |
| Hypo- | 2 | 0 | 0 | 0 | 0 | 0 | 1 | 0 | 0 | 0 | 0 | 0 | 0 | 0 | 1 | 1 |
| albuminaemia | | | | | | | | | | | | | | | | |
| Fibrin D dimer | 0 | 0 | 1 | 0 | 0 | 0 | 0 | 0 | 0 | 0 | 0 | 0 | 0 | 0 | 2 | 0 |
| increased | | | | | | | | | | | | | | | | |

ΔIncluding one patient with dose limiting toxicity

### Example 5

TABLE 10
Results of Different Strategies for Mitigation of Increase
in Liver Parameters in Ongoing Study MT110-101

| Cohort | AST | | ALT | | Bili | | |
|---|---|---|---|---|---|---|---|
| MT110 Dose | (U/I) vs | | (U/I) vs | | vs | | |
| Prophylaxis | BL | +/− | BL | +/− | BL | +/− | Comment |
| I: All pts with same MT110 | 438 | 385 | 437 | 396 | 0.7 | 0.6 | Single |
| dose/corticosteroid schema | | | | | | | administration of |
| Target dose 1 μg | | | | | | | corticosteroids at |
| 1x 100 mg d 0 cort. | | | | | | | treatment start |
| | | | | | | | results in moderate |
| | | | | | | | increase in liver |
| | | | | | | | parameters |
| II: All pts with same MT110 | 8 | 5 | 24 | 16 | 0.3 | 0.2 | Increase of |
| dose/corticosteroid schema | | | | | | | corticosteroid dose |
| Target dose 1 μg | | | | | | | to 2-3x 100 mg on |
| 2-3x 100 mg d 0-2 cort. | | | | | | | days 0 to 2 can |
| | | | | | | | mitigate the |
| | | | | | | | increase in liver |
| | | | | | | | parameters |
| III: All pts with same | 540 | 220 | 510 | 41 | 0.7 | 0.0 | Escalation of |
| MT110 dose/corticosteroid | | | | | | | MT110 dose leads |
| schema | | | | | | | to higher increase |
| Target dose 10 μg | | | | | | | in liver parameters |
| 2-3x 100 mg d 0-2 cort. | | | | | | | that can not be |
| | | | | | | | sufficiently |
| | | | | | | | mitigated by the |
| | | | | | | | corticosteroid dose |
| | | | | | | | of 2-3x 100 mg on |
| | | | | | | | days 0 to 2 |
| IV: All pts with same | 144 | 137 | 154 | 128 | 1.1 | 1.2 | Low MT110 start |
| MT110 start | | | | | | | dose of 3 μg/d |
| dose/corticosteroid schema | | | | | | | combined with the |
| Start dose 3 μg | | | | | | | described |
| 2-3x 100 mg d 0-2 cort. | | | | | | | corticosteroid |
| | | | | | | | schema leads to |
| | | | | | | | only mild increase |
| | | | | | | | of liver parameters |
| | | | | | | | and allows for |
| | | | | | | | further dose |
| | | | | | | | escalation steps |
| V: All pts with same MT110 | 212 | 175 | 273 | 221 | 0.1 | 0.1 | A further increase |
| start dose/corticosteroid | | | | | | | in corticosteroid |
| schema | | | | | | | dose can not better |
| Start dose 3 μg | | | | | | | mitigate the |
| 1 g + 2x500 mg d 0-2 cort. | | | | | | | increase in liver |
| | | | | | | | parameters |
| | | | | | | | compared to the |
| | | | | | | | corticosteroid dose |
| | | | | | | | described in II |
| VI: All pts with same | 69 | 71 | 175 | 138 | 0.4 | 0.3 | A low dose |
| MT110 start | | | | | | | pretreatment as |
| dose/corticosteroid schema | | | | | | | described in IV |
| and 6 μg in step 2 | | | | | | | allows for further |
| Step 2: 6 μg | | | | | | | dose escalation |
| 1 g + 2x 500 mg d 0-2 cort. | | | | | | | that results in only |
| VII: All pts with start dose 3 | 60 | 51 | 102 | 78 | 0.5 | 0.3 | mild increase of |
| μg and 12 μg in step 2 | | | | | | | liver parameters |
| Step 2: 12 μg | | | | | | | |
| 2-3x 100 mg d 0-2 cort. | | | | | | | |
| VIII: All pts with 1 week | 160 | 116 | 192 | 96 | 1.5 | 0.7 | Addition of |
| break between start dose 3 | | | | | | | treatment break |
| μg and 12 μg in step 2 | | | | | | | after start at low |
| Step 2: 12 μg | | | | | | | dose and before |
| 2-3x 100 mg d 0-2 cort. | | | | | | | MT110 dose |
| | | | | | | | escalation results |
| | | | | | | | also in only a mild |
| | | | | | | | increase in liver |
| | | | | | | | parameters |
| IX: All pts with start dose 3 | 305 | 181 | 475 | 216 | 2.9 | 1.7 | Dose-dependency |
| μg and 24 μg in step 2 | | | | | | | of increase in liver |
| Step 2: 24 μg | | | | | | | parameters on |
| 2-3x 100 mg d 0-2 cort. | | | | | | | MT110 dose is |
| | | | | | | | again seen with |
| | | | | | | | higher MT110 |
| | | | | | | | doses; further |
| | | | | | | | mitigation |
| | | | | | | | measures are |
| | | | | | | | necessary to |
| | | | | | | | prevent higher |
| | | | | | | | increase in liver |
| | | | | | | | parameters |
| X: All pts with 1 week break | 630 | 328 | 683 | 39 | 3.8 | 0.8 | One week break is |
| between start dose 3 μg | | | | | | | not enough to |
| and 24 μg in step 2 | | | | | | | sufficiently mitigate |
| Step 2: 24 μg | | | | | | | increase in liver |
| 2-3x 100 mg d 0-2 cort. | | | | | | | parameters at |
| | | | | | | | higher MT110 |
| | | | | | | | doses |
| XI: All pts with start dose 3 | 366 | 347 | 308 | 280 | 1.9 | 2.1 | Stepwise dose |
| μg + 12 μg in step 2 of | | | | | | | escalation leads to |
| cycle 1; escalation step | | | | | | | only moderate |
| 3 = start of 2 | | | | | | | increase in liver |
| Step 3 = cycle 2: 24 μg | | | | | | | parameters; |
| 2-3x 100 mg d 0-2 cort. | | | | | | | optimal lengths of |
| | | | | | | | break period and |
| | | | | | | | size of dose steps |
| | | | | | | | have to be |
| | | | | | | | identified |

1. A method for treating, ameliorating or preventing an EpCAM positive epithelial cancer in a human patient, said method comprising:
(a) administering continually a first dose of an EpCAMxCD3 bispecific antibody for a first period of time to said human patient; and consecutively
(b) administering continually a second dose of said antibody for a second period of time to said human patient;
wherein said second dose exceeds said first dose and wherein said human patient is diagnosed as having, or is suspected of having an EpCAM positive cancer.

2-3. (canceled)

4. The method of claim 1, wherein the route of administration in step (a) or the route of administration in step (b) is intravenous.

5. (canceled)

6. The method of claim 1, wherein said second period of time exceeds said first period of time.

7. The method of claim 1, wherein said first period of time is at least 7 days.

8. The method of claim 1, wherein said first period of time is characterized by an increase of the serum level of at least one liver enzyme up to grade 3 or 4.

9. The method of claim 8, wherein said first period of time persists until the increased serum level of said liver enzyme is decreased to grade 2.

10. The method of claim 1, wherein the first period of time is at least 4 days, provided that the serum level of said at least one liver enzyme is grade 2 or below.

11. The method of claim 8, wherein said at least one liver enzyme is AST and/or ALT and optionally also GGT and/or AP.

12. The method of claim 1, wherein said second period of time persists at least until the CD8+-T-cells of said patient are activated.

13. (canceled)

14. The method of claim 1, wherein said second period of time is at least 2 weeks.

15. The method of claim 14, wherein said second period of time is at least 19 days.

16. The method of claim 1, wherein said first period of time is between 1 and 10 days, and that second period of time is at least 19 days.

17. The method of claim 16, wherein said first period of time is 7 to 9 days and that second period of time is 19 to 21 days

18. (canceled)

19. The method of claim 1, wherein said second dose is therapeutically active.

20-21. (canceled)

22. The method of claim 1, wherein said first dose is such that the serum level of at least one liver enzyme increases to a serum level of grade 3 or 4 and decreases again to a serum level of grade 2 within the first period of time.

23. The method of claim 22 wherein said at least one liver enzyme is AST and/or ALT and optionally also GGT and/or AP.

24. The method of claim 1, wherein said first dose is between 1 and 6 μg/d.

25. The method of claim 1, wherein said second dose is between 10 and 120 μg/d.

26. (canceled)

27. The method of claim 1, wherein said bispecific antibody is a single chain antibody.

28. The method of claim 1, wherein said antibody is MT110.

29. The method of claim 1, further comprising administering a glucocorticoid to said patient.

30. The method of claim 29, wherein said glucocorticoid is prednisone, prednisolone or methylprednisolone.

31. The method of claim 1, wherein said EpCAM positive epithelial cancer comprises gastrointestinal or lung cancer.

32. The method of claim 31, wherein said gastrointestinal cancer is gastric cancer, colorectal cancer, or metastatic variants thereof and said lung cancer is small lung cancer, non-small lung cancer, or metastatic variants thereof.

33. A method for treatment, amelioration or prevention of a an EpCAM positive cancer in a human patient comprising:
(a) administering continually to said human patient an EpCAMxCD3 bispecific antibody such that the serum level of at least one liver enzyme is increased to grade 4 or less (preferably to grade 3) and subsequently decreased to grade 2; and consecutively
(b) administering to said human patient said antibody such that it is therapeutically active.

34. The method of claim 33, wherein the medical condition is an EpCAM positive epithelial cancer.

35-39. (canceled)


SEQUENCE LISTING (7 sequences)

SEQ ID NO: 1 - protein, 10 aa, artificial sequence
  vH CDR1 VH5,7
GYTFTRYTMH

SEQ ID NO: 2 - protein, 17 aa, artificial sequence
  vH CDR2 VH5
YINPSRGYTNYADSVKG

SEQ ID NO: 3 - protein, 10 aa, artificial sequence
  vH CDR3 wt anti-CD3 VH2,3,5,7
YYDDHYCLDY

SEQ ID NO: 4 - protein, 10 aa, artificial sequence
  vK CDR1 VL 1,2
RASQSVSYMN

SEQ ID NO: 5 - protein, 7 aa, artificial sequence
  vK CDR2 wt anti-CD3 VL1-3
DTSKVAS

SEQ ID NO: 6 - protein, 9 aa, artificial sequence
  vK CDR3 wt anti-CD3 VL1-3
QQWSSNPLT

SEQ ID NO: 7 - protein, 515 aa, artificial sequence
  5-10(VL-VH)xanti-CD3(VH(5)-VL(2))
MGWSCIILFLVATATGVHSELVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPLTFGAGTKLEIKGGGGSGGGGSGGGGSEVQLLEQSGAELVRPGTSVKISCKASGYAFTNYWLGWVKQRPGHGLEWIGDIFPGSGNIHYNEKFKGKATLTADKSSSTAYMQLSSLTFEDSAVYFCARLRNWDEPMDYWGQGTTVTVSSGGGGSDVQLVQSGAEVKKPGASVKVSCKASGYTFTRYTMHWVRQAPGQGLEWIGYINPSRGYTNYADSVKGRFTITTDKSTSTAYMELSSLRSEDTATYYCARYYDDHYCLDYWGQGTTVTVSSGEGTSTGSGGSGGSGGADDIVLTQSPATLSLSPGERATLSCRASQSVSYMNWYQQKPGKAPKRWIYDTSKVASGVPARFSGSGSGTDYSLTINSLEAEDAATYYCQQWSSNPLTFGGGTKVEIK